(19) United States
(12) Patent Application Publication
(10) Pub. No.: US 2010/0317093 A1
(43) Pub. Date: Dec. 16, 2010

(54) FLEXIBLE POUCH AND CARTRIDGE WITH FLUIDIC CIRCUITS

(75) Inventors: Marek Turewicz, Westlake Village, CA (US); Paul Pagano, Moorpark, CA (US); Andre' De Fusco, Westlake Village, CA (US); David Sauvageau, Thousand Oaks, CA (US)

Correspondence Address: Weaver Austin Villeneuve & Sampson LLP, P.O. BOX 70250, OAKLAND, CA, 94612-0250, US

(73) Assignee: CYNVENIO BIOSYSTEMS, INC., Westlake Village, CA (US)

(21) Appl. No.: 12/813,282

(22) Filed: Jun. 10, 2010

Related U.S. Application Data
(60) Provisional application No. 61/185,907, filed on Jun. 10, 2009.
(60) Provisional application No. 61/320,629, filed on Apr. 2, 2010.

(51) Int. Cl. C12M 1/34; G01N 33/00; G01N 21/00; G01N 27/00; G01N 27/26; B29C 43/20

(52) U.S. Cl. 435/287.2; 422/68.1; 422/82.05; 422/82.01; 204/600; 435/287.1; 264/241

(57) ABSTRACT

Flexible pouch and flexible cartridge devices for fluid sample processing, related methods of making and using, related manufacturing systems and related instrumentation systems are described. Flexible pouches provide broad advantage in a wide variety of fields by overcoming the need for complex instrumentation, dedicated devices, and relatively high cost in conventional fluid sample devices. Flexible cartridge devices are particularly advantageous in control of fluid handling, rapid adaptation to a number of configurations by the end user, multiple uses for a single configuration, and in cost and ease of manufacture.

### CROSS-REFERENCE TO RELATED APPLICATIONS

This application claims the benefit of and priority to U.S. Provisional Application No. 61/185,907 filed Jun. 10, 2009, and U.S. Provisional Application No. 61/320,629 filed Apr. 2, 2010, the contents of which are incorporated by reference herein in their entireties.

### FIELD OF THE INVENTION

The invention relates generally to fluid processing and, in particular aspects, processing fluids for detection, selection or sorting of particulate moieties. In other aspects the present invention relates to biological fluid processing, detection, sorting or selection of cells, proteins, and nucleic acids. Devices, methods and other aspects are disclosed herein.

### BACKGROUND OF THE INVENTION

Fluid sample processing devices controlling flow generally involve fairly complex fluidic circuits, dedicated devices and associated instrumentation.

With biological sample preparation, complex techniques are used for cell sorting, cell selection (based on surface markers), detection of moieties in a biological sample (such as a rare protein) or screening collections of molecules (for example, screening an aptamer library for its ability to bind to a protein).

Fluids may be stationary or in continuous flow. For stationary liquid samples, the sample (for instance, cells in liquid media), may be placed in a sample preparation container. Analytical or clinical laboratory automation, for example, involves placing samples on microtiter plates having a defined configuration to work in conjunction with automated dispensing and other fluid handling/analytical automated equipment. The sample container itself performs no analytical function, but is merely a receptacle designed to hold liquid sample to be analyzed, transferred or otherwise disposed by coordinating instrumentation. This, too, involves dedicated instrumentation and adapted robotic design (for automation, for example).

Continuous flow processing is also known. Cell sorting using flow cytometry is available, for example, based on fluorescence, or magnetic activated sorting. Cell cytometry devices are typically based on moving a suspension of cells in a liquid stream through a sensing zone. Cells to which a detectable label is attached (such as a fluorescent or magnetic) are sensed, and then sorted away from the remaining sample by deflection, typically. Flow cytometric sorting is suited to cell sorting because selection of cells or particles may be based on multiple, simultaneously measured characteristics. Flow cytometry is also used to sort out relatively rare cells and provides for a high purity of the sorted cells. Flow cytometry, however, is relatively complex, requires expensive instrumentation and skilled personnel, and nevertheless requires relatively long times to obtain large numbers (millions) of sorted cells. See generally, Hoffman, Robert A. and David W. Houck, “Cell Separation using Flow Cytometric Cell Sorting,” Chapter 11, pages 237-269, in: Cell Separation Methods and Applications, Diether Recktenwald and Andreaus Radbrusch, eds., Marcel Dekker, Inc. 1998.

Microfluidic devices are becoming increasingly more available, and can provide distinct advantages over flow cytometry apparatus, rigid columns, tubes and microtiter-plate based devices, each of which are widely used for separations and analysis of biological materials. Small scale and increased sensitivity, combined with ease of use, provide distinct advantages in a variety of applications. Microfluidic devices are available for such purposes as on-device protein purification, rare cell separation, and screening for rare molecules (such as proteins or aptamers) in a sample. See, for example, J. Qian, X. Lou, Y. Zhang, Y. Xiao, H. T. Soh, “Rapid Generation of Highly Specific Aptamers via Micromagnetic Selection” Analytical Chemistry (2009); U. Kim and H. T. Soh, “Simultaneous Sorting of Multiple Bacterial Targets Using Integrated Dielectrophoretic-Magnetic Activated Cell Sorter” Lab on a Chip (2009); Y. Liu, J. D. Adams, K. Turner, F. V. Cochran, S. Gambhir, and H. T. Soh, Controlling the Selection Stringency of Phage Display Using a Microfluidic Device. Lab on a Chip (2009); X. Lou, J. Qian, Y. Xiao, L. Viel, A. E. Gerdon, E. T. Lagally, P. Atzberger, T. M. Tarasow, A. J. Heeger, and H. T. Soh, “Micromagnetic Selection of Aptamers in Microfluidic Channels,” Proceedings of the National Academy of Sciences, USA, (2009).

To date, however, microfluidic devices contain structural components directly formed on the device itself “Lab on a chip” devices, for example, typically involve precision fluidic chambers, interconnecting pumps and valves, and fluid injection comprising a fluidic circuitry. Introduction to Microfluidics, id., at 16-17. These features and functions are generally accomplished using a rigid platform, commonly called a cartridge, upon which analytical components are manufactured. Microfluidic devices use rigid materials in order to compartmentalize different functions such as incubation, interrogation, and waste. Rigid devices are also designed to be interoperable with processing and detection devices, such as automated pneumatic and mechanical pumping devices (that are operable with particular valve configurations on cartridges) or optical readers.

Manufacturing a microfluidic cartridge requires precise geometries, precision components and assembly methods, and built in precision valves, for example. For cartridge producers, this requires a relatively high capital cost and skilled personnel. Even larger systems, for example conventional magnetic bead separation columns, are made of rigid materials and are not adaptable to various configurations using a single cartridge.

Moreover, there are manufacturing constraints in configuration. Lithography and etching technologies may be used to manufacture the precise design for desired microfluidic flow. At a reduced cost, one may use injection molding for preparation of a rigid base having a particular configuration. Preparing the base alone, however, leaves the chambers and channels (in which the liquid flows) open. To enclose the device, one may then seal a top layer of similarly rigid material using laminate, heat, acoustic or laser (to adhere a top layer for a sealed device).

Thus, rigid materials, such as glass, vinyls or other conventional plastic polymers, may be used for “lab on a chip” and other devices. Although there may be some elasticity or plasticity (such as by using a thin laminate as a transparent cover), the device surface must be configured, whether by injection molding or by lithography (or etching or particle deposition technologies). The cartridge design (channels, inlets, compartments and other fluid flow paths or containment areas) is indelibly configured. Interconnection elements, such as ports use for inlet or outflow, or for pressurized flow/stoppage, may be attached, or formed as part of the injection molded design (for example). Other elements, such as electro-, mechanical, or various sensors are similarly in dedicated location. Only with great effort (for example, re-casting) can the cartridge be re-configured for a different flow path with a different microfluidic design. Various flexible detection devices are reported, such as flexible biochips with gold electrode patterns were fabricated on thin polyethylene naphthalate (PEN) foils using photolithography. Peter et al., “Flexible Biochips for Detection of Biomolecules,” Langmuir 25:5384-5390 (2009) DOI: 10.1021/1a8037457 Publication Date (Web): Mar. 30, 2009. These are noted to have improved manufacturing convenience for large area roll-to-roll manufacturing although the electrode pattern itself is dedicated on the flexible device.

Other devices for characterization and/or isolation of components of biological samples include rigid columns, for example, columns that isolate a biological component from a complex mixture using magnetic beads with attached biomarkers that selectively bind to the desired moiety to be isolated from the complex mixture. Although these devices represent an advance, there are drawbacks. For example, the columns are rigid bodies and essentially serve a single function, that is, a biological fluid mixed with magnetic particles that have an affinity for a particular species in the sample is eluted through the column which contains media that creates a localized magnetic field gradient which traps the magnetic particles with the selectively attached species. There is no ability to adapt the column to multiple uses, there is a single inlet and a single outlet, no fluidic circuitry is available. So, although useful, these columns have limitations.

Regardless, the current technology requires sunk costs, skilled personnel, and dedicated tooling and fixtured devices that are difficult to reconfigure after initial manufacturing. Moreover, high throughput applications, such as processing large numbers of samples, necessarily are limited by automation adapted for a rigid cartridge (as in the case of the microfluidic devices).

As such, there is a need for a fluid sample handling device that may be configured for liquid sample preparation and optionally particle detection, sorting or analysis, that may be manufactured in large volumes at low cost, with little capital investment for design configuration set up, and with ease of use, use in high throughput applications, and adapted to a variety of uses and instrumentation.

### SUMMARY OF THE INVENTION

Flexible fluid analysis devices, including flexible pouches and flexible cartridges devices, for fluid sample processing, related methods of making and using, related manufacturing systems and related instrumentation systems are described. Flexible pouches provide broad advantage in a wide variety of fields by overcoming the need for complex instrumentation, dedicated devices, and relatively high cost in conventional fluid sample devices. Flexible cartridge devices are particularly advantageous in control of fluid handling, rapid adaptation to a number of configurations by the end user, multiple uses for a single configuration, and in cost and ease of manufacture.

One embodiment is a flexible fluid analysis device, having a unitary body, including: (i) at least one reservoir in fluid communication with; (ii) a first fluid channel also in fluid communication with; (iii) a mixing chamber, the mixing chamber also in fluid communication with; (iv) a second fluid channel which is also in fluid communication with; (v) an outlet for draining fluids from the mixing chamber.

One embodiment is a flexible pouch device with fluidic circuitry. Flexible pouches of the invention provide broad advantage in a wide variety of fields by overcoming the need for complex instrumentation, dedicated devices, and relatively high cost in conventional fluid sample devices.

Another embodiment is a flexible cartridge device for fluid sample processing. Flexible cartridge devices of the invention are particularly advantageous in control of fluid handling, rapid adaptation to a number of configurations by the end user, multiple uses for a single configuration, and in cost and ease of manufacture.

Other embodiments include apparatus and instrumentation to manipulate flexible pouch and/or cartridge devices described herein as well as methods of manufacture of flexible pouch and cartridge devices.

The present invention may be used in a broad array of applications, including (but not limited to) biological fluid sample preparation and analysis, separation of rare molecules (such as nucleic acids or cells) from a complex mix, chemical library screening, health care related diagnostics (as in a clinical laboratory context, for example), environmental testing or monitoring, consumer products and food quality control aspects. The present invention has correspondingly broad industrial utility as is described more fully herein.

Also, in various aspects, provided are kits (including prefilled devices), methods of use, manufacturing systems and instrumentation systems, and other aspects as more fully described herein.

### BRIEF DESCRIPTION OF THE DRAWINGS

FIG. 1 is a schematic of a flexible pouch manufactured from a polyethylene bag, as described more particularly in Example 1.

FIG. 2 is a schematic illustrating a flexible pouch system, 200, of the present invention.

FIG. 3 is a block diagram of a flexible polymer pouch fluid analysis device.

FIG. 4 is a block diagram of a clamshell nest (holder) system prophetically containing a flexible pouch or cartridge fluid analysis device of the present invention.

FIG. 5 is a schematic illustration of a fluidic circuitry configuration for flexible pouch or cartridge devices described herein.

FIGS. 6A and 6B depict various views of a flexible cartridge fluid analysis device of the invention.

FIG. 6C depicts various views of another flexible cartridge fluid analysis device of the invention.

FIGS. 7A-7C are process flows in accord with methods of the invention.

FIGS. 8A-8D are schematics of a clamshell device of the invention for use with the flexible cartridge as described in relation to FIGS. 6A and 6B.

FIGS. 9A-9L are flow diagrams showing valving and operational processes used in conjunction with the flexible cartridge device as described in FIGS. 6A and 6B and the process flow in FIG. 7C.

### DETAILED DESCRIPTION OF THE INVENTION

The present invention stems from the observation that a flexible pouch, for example, of the type used in the packaging field (for food or consumer goods, for example) may be adapted as a device for fluid sample processing. As well, blow molding allows formation of flexible cartridges that include fluidic circuitry and are adaptable to many uses, particularly when combined with external manipulation, for example, using apparatus described herein.

Aspects of the invention relate to apparatus and methods for manipulating a flexible pouch or cartridge using external forces in order to create fluidic circuits that have, for example, reservoirs, reaction chambers, delivery conduits, and the like. In one context, a flexible pouch, for example with a unitary volume i.e. a “featureless bag”, can be manipulated by external forces to create a fluidic circuit where previously there was none. Put another way, in the invention embodies defining fluidic circuitry entirely by how applied external manipulation configures an otherwise featureless or limited feature flexible pouch or cartridge. The opposite end of the spectrum would be where all components of a fluidic circuit are built in, or integral to, the structure of a fluidic device, i.e. a completely “rigid” device.

In one embodiment, a featureless bag, as described above, is inserted into an apparatus, for example a clamshell type apparatus (e.g. as described in more detail below with respect to various embodiments), that engages the featureless bag in order to create a fluidic circuit within the featureless bag as a result of the engagement with the featureless bag. That is, upon engagement with the clamshell, all or a portion of the featureless bag is transformed into a fluidic circuit. For example, an external clamping or molding force creates fluidic channels, reservoirs, etc. that reflect the shape of one or more clamps or molds in the apparatus. In this embodiment, the featureless pouch is given features only when in the clamshell or other featured hollow support structure of an apparatus that engages the featureless pouch. Such apparatus will also have actuation mechanisms for creating and controlling valves in the fluidic circuit, pumping fluids within the fluidic circuit and manipulating species in a fluid sample within the fluidic pouch, for example, external magnetic fields, etc. as described in more detail below. One embodiment is an apparatus that can be configured to create multiple fluidic pouch configurations from a featureless pouch, for example, by switching out modular molds and/or clamps. A clamshell type apparatus is particularly useful for implementations of this embodiment, because opposing plates, for example, with premilled molds, can be employed along with appropriate actuators and external force generators, for example magnetic fields, to create and manipulate a fluidic circuit for analysis of, including isolation of a target species from, a fluid sample.

Although aspects of the invention include creating a fluidic circuit from a featureless bag, other embodiments of flexible pouches and/or cartridges of the invention as described herein typically have some components of the fluid circuitry, or at least are configured with precursors of fluidic circuitry to aid in creating a desired fluidic circuit when one or more external manipulations are applied. For example, a flexible pouch or cartridge of the invention can have pre-formed, for example via blow molding, larger volumes interconnected by conduits. These conduits serve as fluid communication channels between various volumes of the pouch or cartridge. These features are typically not particularly useful without application of external forces to create a functional fluidic circuit for a desired outcome, for example cell separations, protein purifications and/or molecular separations and/or reactions.

A few non-limiting examples of application of external forces for manipulation and/or creation of a fluidic circuit are: 1) applying a clamping or molding force to create a fluidic circuit, 2) applying external force to one or more conduits to close them shut during particular operations and thus create valves of a fluidic circuit, 3) applying external tensile force to one or more volumes in order to pump fluid from a volume, through a conduit and into another volume, out of the device, mix fluids together, etc., 4) applying external magnetic (or acoustic or vibrational) force in order to manipulate particles within a volume of the fluidic circuit, deform or pinch shut a portion of the pouch via pulling or pushing a magnetic body against the pouch, and 5) applying a pneumatic force, vacuum or pressure externally, to manipulate fluids within the pouch and/or create components of the fluidic circuit, such as with gas knifes to pinch shut a portion of the device or deform a volume in order to pump or mix fluid.

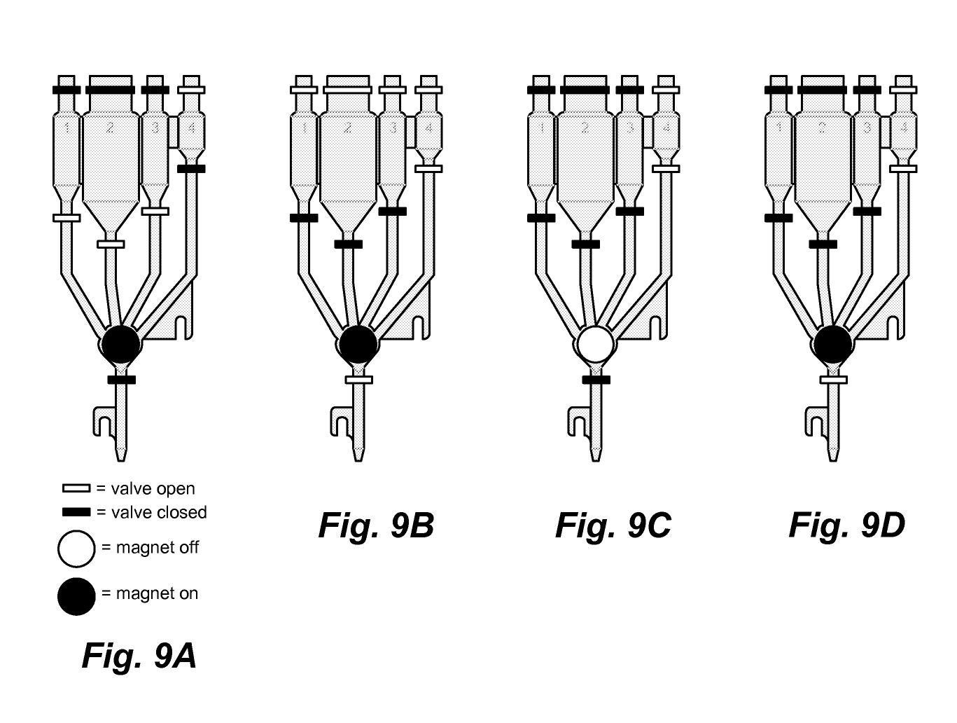

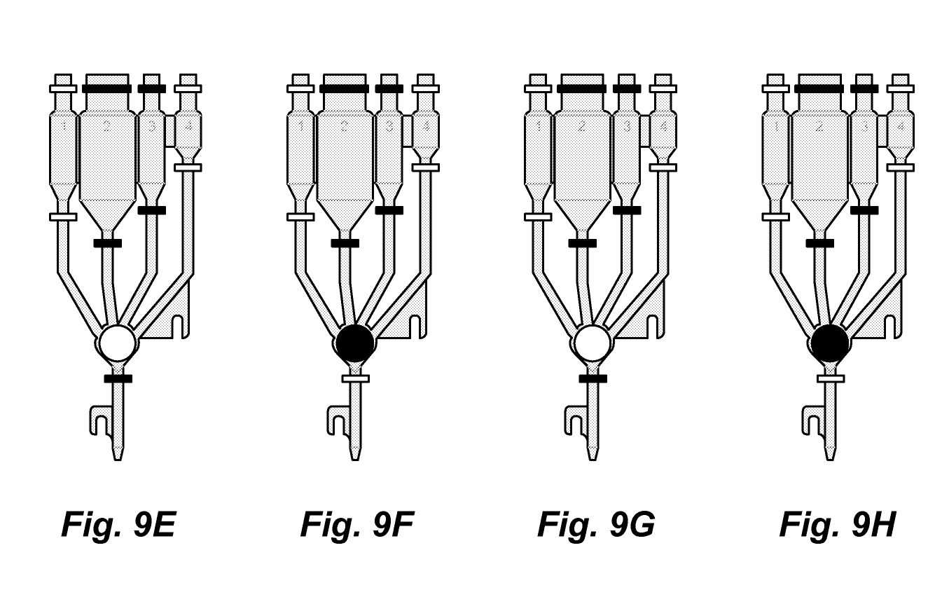

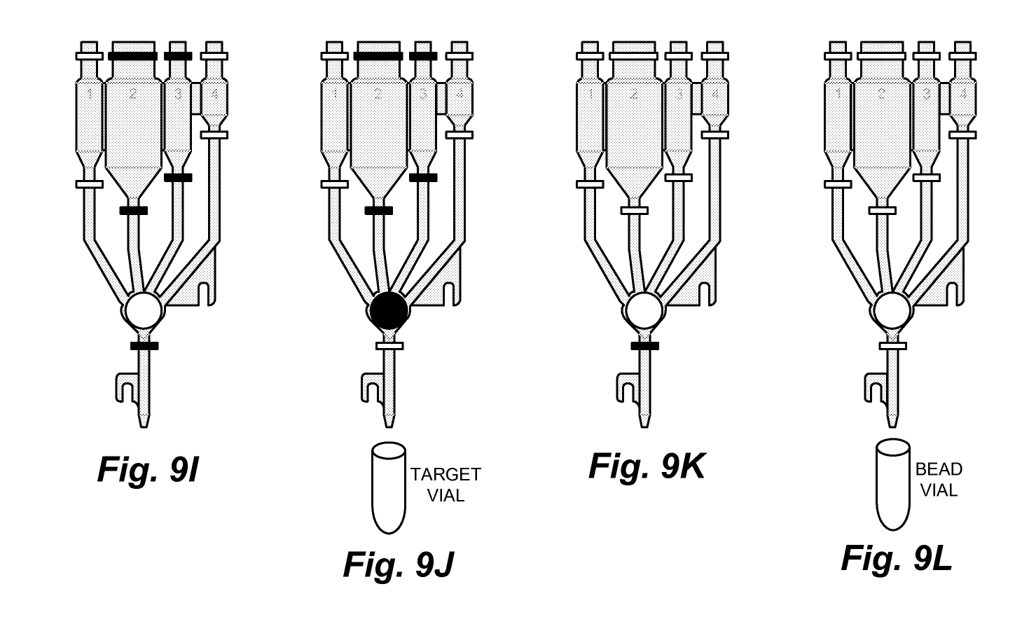

Along with forces applied to the exterior of flexible fluidic devices described herein, there may also be internal forces applied. For example a pneumatic force such as a gas pressure or a partial vacuum, or a hydraulic force, such as fluid pressure can be used to move fluids within the device. In one example, alternating application of gas pressure and partial vacuum are applied to one or more inlets and/or outlets in order to move fluids within a mixing chamber, for example as described in relation to FIGS. 9A-9L. In another example, gas pressure is used to push against capillary action in order to remove fluids from a device. In yet another example, a buffer solution is used to push a fluid from one chamber to another in a flexible fluid analysis device.

Flexible pouch devices are particularly advantageous in ease of manufacture, control of fluid handling/flow (for processing within the pouch), rapid adaptation to a number of configurations by the end user, and ability to process large numbers of devices by rolling automation design, blow molding and the like. Particular aspects and advantages are readily apparent from description provided herein.

Importantly, constraints of rigid structures are avoided. As indicated above, some current devices are microfluidic cartridges (see, for example, PCT/US2005/042798, PCT/US2007/022105 PCT/US2007/022118, PCT/US2008/006599, and PCT/US2008/074107, herein incorporated by reference). The microfluidic devices and methods disclosed in the above publications are practicable for a broad range of applications, and, in practice, devices are manufactured as a rigid cartridge. With rigid microfluidic cartridges, for example, fluids may be pumped pneumatically or mechanically via integrated ports. The present flexible pouches may be used in conjunction with rolling or other tensile pressure to effectuate fluidic movement, and/or for example valving in the device, as well as pneumatic or other flow-controlling force. Because the devices of the invention are flexible, configurations for pumping, valving and the like are dynamic, that is, they can be varied within a single device depending upon the needs of the methods employed using the flexible pouch and/or cartridge device.

Moreover, the present flexible pouch design provides manufacturing advantages in eliminating much of the cost and complexity, particularly for “lab on a chip” applications. For producers, the flexible pouch technology is inexpensive (as compared to relatively rigid microfluidic cartridges manufactured by injection molding or lithographic techniques), easily configurable, and can incorporate a variety of designs and materials.

Exemplary blow molding techniques and materials suitable for embodiments of the invention can be found, for example, in “Plastic Blow Molding Handbook by N.C. Lee (1990),” and “Blow Molding Handbook by D. Rosato, D. Rosato and D. Mattia (2003),” both of which are herein incorporated by reference in their entireties.

Flexible cartridges of the invention are made, for example, via blow molding. Where, for example, a small mass of thermoplastic material is heated and blown to conform to a preformed mold. In this way, flexible cartridge devices may be manufactured in large numbers with relative ease, as described in more detail below. Blow molding has two fundamental processes. First, a preform (or parison) of hot plastic resin in an oblong shape is created. Second, a pressurized gas, for example air, is used to expand the hot preform from within, like blowing up a balloon, and press it against a mold cavity. The pressure is held until the plastic cools. Once the plastic has cooled sufficiently to maintain the molded shape, the mold opens and the molded device is ejected. Blow molding can be, for example, extrusion, injection and/or stretch type blow molding. One advantage of using blow molding, besides ease and low cost of manufacture, is that internal chambers and channels are all relatively smooth which aids in fluid flow through the device as opposed to conventional rigid devices with acute angles or sharp interior edges where fluid can collect and resist flow due to capillary action and surface phenomenon. In addition, blow molding can create a finished device in a single step with no secondary operation needed to enclose the channels. For example one does not need to seal the channels by lamination, heat, laser, acoustic, or adhesive.

Flexible pouches can also be made, for example, by molding and cutting from a single source of material, for example a roll of polypropylene film. Manufacturing of flexible pouches typically involves a single machine that fills, configures and seals the pouch. Pouches are manufactured from rolls of flexible polymer material (for example). Thus, flexible pouch devices may be manufactured in large numbers with relative ease.

For consumers, the flexible design allows for high throughput automated processing and handling of flexible pouch devices where the devices are flexible and not subject to breakage with shear stress. Although the rigid cartridge type devices may be processed using automation instrumentation, the present flexible pouch devices may be processed using existing roller-type technologies. Thus, the present flexible pouch devices permit fluidic sample handling, including internal fluid flow manipulation, using tensile pressure via roller (or other mechanical) high throughput means.

Moreover, in some aspects, the present flexible pouch design permits one to manually control precise fluid movement by placing tensile pressure on the flexible pouch itself. This permits use in relatively remote regions, areas without reliable electricity, etc. Individuals conducting such fluidic movement need not be skilled operating automation devices. Thus, the present flexible pouch devices provides for sophistication application with extremely low cost and no instrument training skills involved.

### Terminology:

Unless otherwise defined, scientific and technical terms used in connection with the present invention shall have the meanings that are commonly understood by those of ordinary skill in the art.

The term “pouch” is generally used in its ordinary meaning in the product packaging field, in the context of the further description provided herein. Generally, the term “pouch” denotes flat (for example, pillow, four-side seal and three-side seal) and stand-up pouches such as those employed in food, beverage and nonfood applications. The term “pouch” also encompasses those that are resealable, aseptic, vacuum, retort, shaped and stick or spouted pouches (which can be produced in flat or stand-up varieties). Although the extant consumer product pouches are particularly suited for the present invention, bags and sacks, as well as non-packaging pouch applications and air cushioning pouch packaging systems are similarly encompassed to the extent these can be configured with fluidic circuits as described herein.

“Flexible Fluid Analysis Device” means a device for fluid handling and analysis where the entire device is flexible. Examples of a flexible fluid analysis device are a flexible pouch and a flexible cartridge as defined in more detail herein. Flexibility may be understood as a mechanical/functional property of the active components of the device. Specifically, active or deformable components of the device such as valves should be sufficiently non-rigid to reversibly undergo deformation. Thus, the active components should not break or otherwise make the device unusable in response to repeated deformation of a type necessary to actuate the active components of the device. All non-active components of the device should have a mechanical rigidity that is similar to that of the active components. Thus, the entire device is non-rigid. In a non-limiting embodiment, no portion or component of the device has a shear modulus of greater than about 0.1 GPa.

“Flexible Pouch” fluid analysis device means a device for fluid handling and analysis where the entire device is flexible, that is, there are no rigid or semi-rigid components in the fluidic circuitry. A flexible pouch fluid analysis device has fluidic circuitry by virtue of molding fluidic circuitry into layers of flexible material and/or by external manipulation of the pouch. A flexible pouch requires some support during operation, for example, the pouch can be oriented horizontally on a surface and manipulated thereon or suspended vertically and manipulated from either side. A flexible pouch fluid analysis device has fluidic circuitry that is manipulated during operation. This fluidic circuitry can be entirely temporary, for example, one or more molds are applied to a featureless bag to create a flexible pouch with fluidic circuitry. Operations are carried out within the fluidic circuitry by pumps, magnets and the like which are part of an apparatus for carrying out operations on the pouch. In one embodiment, the molds and actuation components are part of a single device for forming a flexible pouch, for example, from a featureless bag and manipulating the flexible pouch once created. Once the one or more molds are disengaged, the flexible pouch returns to a featureless bag, the fluidic circuitry is lost. The fluidic circuitry can also be permanent, for example, one or more molds are applied to a featureless bag (or layers of material) in order to form, for example via heat lamination and/or by use of appropriately-applied adhesive, the fluidic circuitry of the flexible pouch. Once formed the flexible pouch can be manipulated as described. Thus flexible pouches need some support, first a mold, to form the fluidic circuitry, either temporary, permanent or some combination thereof, and second support necessary to manipulate the pouch, such as a support surface or support structure to suspend the pouch during operation.

“Flexible Cartridge” fluid analysis device, means that the device is not rigid to the point where deformation would break or otherwise make the device unusable. A flexible cartridge differs from a flexible pouch, in that typically all, but at least some, of the features return substantially to their original shape after an applied force is removed without the need for a mold or other support structure. However, like the flexible pouch, the entire device is flexible, as opposed to devices which may have one or more flexible components integrated with rigid components. For example, one flexible cartridge described herein has a blow-molded unitary body that holds its shape but otherwise is flexible and can be reversibly deformed. Generally, flexible cartridge devices of the invention allow pressure or other forces to be applied to the device and allow deformation, for example to valve a particular area of the device or pump fluid from a portion of the device, without damaging the device. In one embodiment, automated instrumentation, for example a clamshell device as described in more detail herein, manipulates the flexible cartridge via pistons, pumps, and other actuators and the flexible cartridge can be reversibly and operably deformed in one or more regions, simultaneously or not, repeatedly; for example, hundreds of times, thousands of times, or even tens of thousands of times while maintaining functionality.

Conventional relatively rigid devices are not meant to be deformed in this way, therefore although conventional devices may exhibit some ability to deform without breakage, they are not designed for the purpose of engaging with, for example, pistons, stepper motors or actuators that deform the device for the purposes of fluid movement, mixing, valving and the like. Another difference between conventional rigid fluid analysis devices and the flexible cartridge devices of the invention is that the invention allows for adaptability in valving, pumping and pneumatic action on a single device. For example a single device can be used to carry out many different fluid analysis protocols by changing how the device itself is manipulated, for example, by timing and location of force by external pistons, rollers, magnetic fields, actuators and the like. The term “cartridge” is meant in the conventional sense, that is, a container for, in this case, liquid made for ready insertion into a device or mechanism that manipulates the container, in this case for fluid handling and analysis. Thus, another distinction from conventional devices, is that flexible cartridges of the invention, in certain embodiments, are meant to be placed in a mechanism where the mechanism applies one or more external forces to the cartridge as part of manipulating the fluid within the cartridge for analysis of the fluid. The one or more external forces include at least one mechanical force that reversibly deforms the cartridge during fluid analysis.

Fluid mechanics terminology: The various terms describing fluid mechanics (including micro fluidics), are used in their conventional technical meanings The term “fluid” and the term “liquid” are used synonymously herein to refer to substances that flow and optionally take the shape of a container. Under some circumstances, there may be gaseous or solid substances that flow. For example, finely granular materials may flow.

The term, “fluidic circuit” refers to a configuration of fluidically interconnected functional areas. The present flexible pouch devices comprise fluidic circuits. As described in more detail herein, functional areas include reservoirs or compartments and channels through which fluids may flow. The channels may be optional, for example, where two compartments are directly fluidically connected as by an adjoining wall. Two reservoirs or chambers may be reversibly fluidically connected, such as via adjoining wall that may be sealed and opened, or may be porous, allowing only certain size particles to flow through. A skilled practitioner will appreciate the numerous configurations possible for the present fluidic circuitry. Apart from configuration, there are similarly wide variety of choices to integrate fluidic circuits, such as the flow control structural elements described herein.

Particle sorting terminology: The term “moiety” as used from time to time herein denotes a “portion” and includes reference to a particle. A “particle” refers to a small object that behaves as a whole unit in terms of its transport and properties. The term “analyte” can be a “moiety” or a “particle” and is used in its ordinary meaning as a substance the presence of which is detected, or a characteristic of which is measured, in an analytical procedure.

Biological and biochemical terminology: Where specific categories of molecules are discussed, such as nucleic acids or proteins, synthetic forms are included, such as mimetic or isomeric forms of naturally occurring molecules. Unless otherwise indicated, modified versions are similarly encompassed, so long as the desired functional property is maintained. For example, an aptamer selective for a CD34 cell surface protein includes chemical derivatives (for example, pegylated, creation of a pro-form, derivatized with additional active moieties, such as enzymes, ribozymes, etc.)

General terminology: In this application, the use of the singular includes the plural unless specifically stated otherwise. In this application, the word “a” or “an” means “at least one” unless specifically stated otherwise. In this application, the use of “or” means “and/or” unless specifically stated otherwise. In the context of a multiple dependent claim, the use of “or” refers back to more than one preceding independent or dependent claim in the alternative only. Furthermore, the use of the term “including,” as well as other forms, such as “includes” and “included,” is not limiting. Also, terms such as “element” or “component” encompass both elements and components comprising one unit and elements or components that comprise more than one unit unless specifically stated otherwise. Where a “skilled practitioner” is referenced, this refers to an ordinary skilled practitioner in the art to which the subject matter pertains, in context, unless otherwise noted.

### General Considerations:

General considerations for making and using the present invention include the overall device configuration, materials, manufacturing systems, instrumentation systems, and applications. Particular embodiments including working examples are also presented. Prophetic examples are also included below.

Flexible fluid analysis devices as described herein may serve as automated sample preparation systems, for performing a series of operations, at least some of which are conventionally conducted manually or by disparate instruments in a laboratory. Often a flexible fluid analysis device integrates various sample preparation functions in a unitary or closed system which provides one or more inlets for raw materials and one or more outlets for purified or otherwise modified materials. Examples, of the purified materials include molecules such as proteins or nucleic acids, virus, and cells such as mammalian cells and bacteria. The purified material may be homogenous or a fraction of the raw input material. Types of automated functions include (1) preparation or modification of a raw sample to facilitate separation, (2) actual separation of target from non-target components of the sample, (3) modification of the target components, and (4) delivery of the target components to a receptacle. Any combination of these functions may be performed in the flexible fluid analysis devices described herein. In some cases, the sample preparation or modification may involve one or more of the following operations: labeling the sample, washing the sample, and incubating the sample. In some cases, the target modification may involve removing a label, chemically or biologically modifying the target, and lysing the target. Additional operations include the actual separation and of the target from the non-target components and eluting the target. In a specific embodiment, a flexible fluid analysis device is used to label a sample, wash the sample, separate the target from the sample, and elute the target, all in an automated fashion. The embodiment may also include an incubation operation before or after washing. Of course, other sequences of operations are within the scope of the invention and some of these will be set forth below.

Configuration: The present flexible pouch devices with fluidic circuits for sample manipulation and analysis may be configured any number of ways depending on the use to which the device will be put. For example, the present flexible pouch device can be used in any way that current rigid fluid handling devices are used. General considerations include the desired manufacturing method systems, the desired use, and the desired related instrumentation (if any).

One of ordinary skill in the art will consider pouch device geometries in conjunction with sample size and partitioning requirements, means for controlling fluid flow direction, path, and rate; means for selecting or sorting particulate matter (if desired), as well as adaptation with instrumentation required.

Use of Flexible Pouches generally and manufacturing advantages: Readily available flexible pouches such as those used in the product packaging field, may be so adapted for the present flexible pouch devices. In the product packaging field, flexible pouches are used for transport or storage of their contents—and typically not as a functional instrumentality in and of themselves. Flexible pouches are also used in large scale biotechnology fermentation. Although disposable reaction vessels have been used for growing cells in culture, for example, “wave bioreactor”, disposable cell culture bioreactors are generally not configured for processing of biomaterials, such as protein purification. One typically must then perform protein purification steps as an additional process after obtaining a cell pellet (for example) and washing, filtering and other processing steps performed while transferring the desired materials among various containers.

The present flexible pouch devices are a single unit which not only contain fluid sample (as, for example, cells in culture, proteins to be purified, molecules to be separated and/or reacted), but also provides means to move all or a portion of the fluid sample out of a containing module, and to another area. Alternatively or additionally, the present flexible devices are a single unit providing means for sorting particles from a fluid suspension. This is described further herein. Flexible pouches may have other physical parameters as will be apparent.

Flexible pouch technology, because it allows for plastic or elastic configuration of the device itself, may facilitate adaptation for particular needs. One may use external pressure for adapting internal configuration (such as using a releasable pressure mold), for mixing contents (such as using pressure exerted by hand for mixing labeling beads with particles in suspension), and various sorting. Thus, depending on the materials and other considerations recognizable to one of ordinary skill in the art, one may suitably modify a flexible pouch device of the present invention during the course of use.

Expansion and contraction may permit moderation of internal gaseous or liquid pressure. Further, the device may be pre-filled, pre-sterilized, or treated in situ in accordance with selected configurations and materials.

As mentioned above, flexible cartridge fluid analysis devices of the invention are typically manipulated by one or more external forces, for example, pumps, pistons, actuators, rollers and the like. In one example, pistons are used for at least one of pumping fluid within the device and valving off particular sections of the device during analysis. In addition, the fluid sample or components thereof can be manipulated with, for example, magnetic fields while residing or flowing, for example circulating, in the cartridge fluid analysis device.

One embodiment is a blow molded flexible cartridge for manipulation of a fluid sample, wherein the manipulation includes at least one of a cell separation, a protein purification and a molecular separation.

Size: The overall size of the present device (pouch or cartridge) may be determined according to the use to which the device will be put, the coordinating instrumentation and related devices, and the practical requirements of space, storage stability (for example, of contained reagents), convenience of use, and other considerations that will be apparent to a skilled practitioner.

Apart from any limitations on practicable application, the lower limit of size is constrained predominantly by manufacturing methods. For example, if one desires nano-electronic components, one may have such components integrated using micro-lithographic techniques. For microfluidic applications, one may seek to proportions limiting turbulence in fluid flow and optimizing laminar flow in a desired path. The present devices can be configured for use with sample volumes of 1-10 μL, 10-100 μL, 100-1000 μL, 1000 μL-100 ml, for example.

In some embodiments the present devices can be configured for use with larger sample volumes from between about 100 ml to 1 liter, and in some cases multi-liter scale. For example, the flexible cartridge device as described in relation to FIGS. 11A and 11B can be configured to hold such volumes due to some structural strength related to its configuration and material make up. In one embodiment, the reservoirs of the flexible cartridge device of the invention are configured to hold between about 0.1 ml and about 10,000 ml, in another embodiment between about 0.1 and about 1,000 ml, in another embodiment between about 0.1 ml and about 100 ml, in another embodiment between about 1 ml and about 50 ml, in yet another embodiment between about 1 ml and about 25 ml.

The present devices may be scalable to virtually any size, again with practical considerations such as fluid mechanics, materials used and the desired application. For use as a bioreactor, for example, the present flexible pouch design may provide advantage in scaling up culture volumes. Fluidic circuitry may provide means for separation, isolation, detection, or other techniques relating to rare molecules or rare cells in culture. For example, if the present flexible pouch device is used to capture relatively rare stem cells, one may then grow the stem cells so captured in situ with a suitably configured pouch. If one seeks to grow cells in culture to prepare a desired expression product, such as a protein, fluidic circuits may be designed to culture a sufficient volume of cells, and then lyse, and select the desired protein (for example). Because the flexible pouch has the advantages of disposable bioreactors it may be concomitantly used as a disposable bioreactor in addition to its use of fluidic circuitry for purifying and isolating a target protein (for example).

### General Structural Elements:

In general, the present flexible pouch devices function to provide fluidic movement to effectuate a particular application, beyond simple fluid containment or storage.

Structural features provide for this. The present flexible pouch devices comprise one or more access ports, one or more reservoirs, and one or more channels providing fluidic communication between or among access ports and reservoirs. Other structural components are also described.

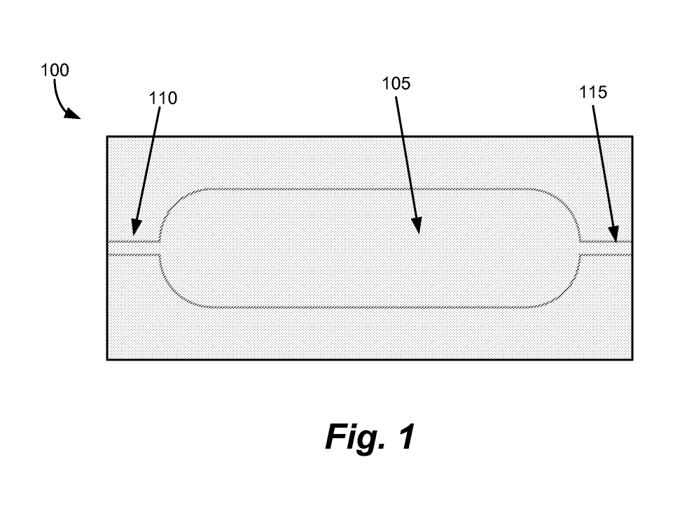

Access ports: In general, depending on the overall configuration and materials, the present flexible pouch devices will have at least one access ports where materials are admitted or exited, and at least one reservoir for containing a fluid. For example, FIG. 1 illustrates a flexible pouch device, 100, of the present invention where there are two access ports, 110 and 115, located at opposing ends of a reservoir, 105. Device 100 has a unitary body manufactured by applying, for example, opposing premilled molds against each other with overlapping layers of plastic so that the device features are formed by virtue of pressing together and fusing the layers together except for where the features are desired. Access ports, used for fluid (or other material) entry or exit, may be configured to operate with other apparatus or instrumentation as part of an overall system. Access ports may connect with external environment, such as by providing a way for fluid fill or fluid exit. For fluid fill, the access port may be configured for fill via syringe, pipette, or by automated filling instrumentation. Fluid exit may be, for example, waste disposal. Or, exit may be part of a positive selection scheme, whereby particulate matter in a suspension is selectively captured. Various types of external interconnects for access ports may be used, such as tubing studs, hose barb connections, O-ring connections, or other external types of interconnections.

Access ports, rather than being an external interconnection, may alternatively be in fluid communication with another portion of the device, such as a separately enclosed reservoir. Functionally, the same purpose is served, for example, fluid fill or fluid partitioning (exit).

Devices of the present invention can have one or more than one access port for a variety of functions, such as for introducing a number of different fluids or for exiting separate moieties, and each may optionally be connected with the external environment or with another portion of the device.

The present devices may be configured for static sample handling or for continuous flow, or for intermittent flow (or other flow patterns).

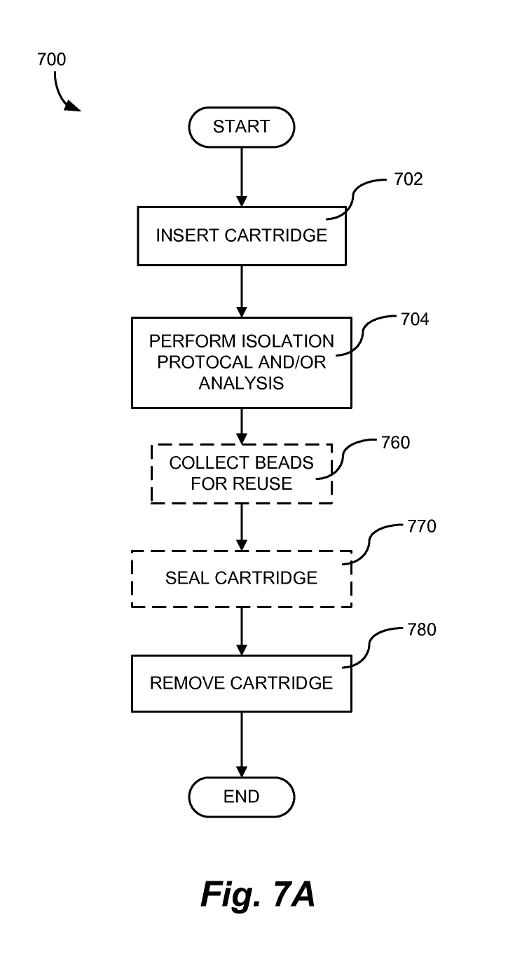

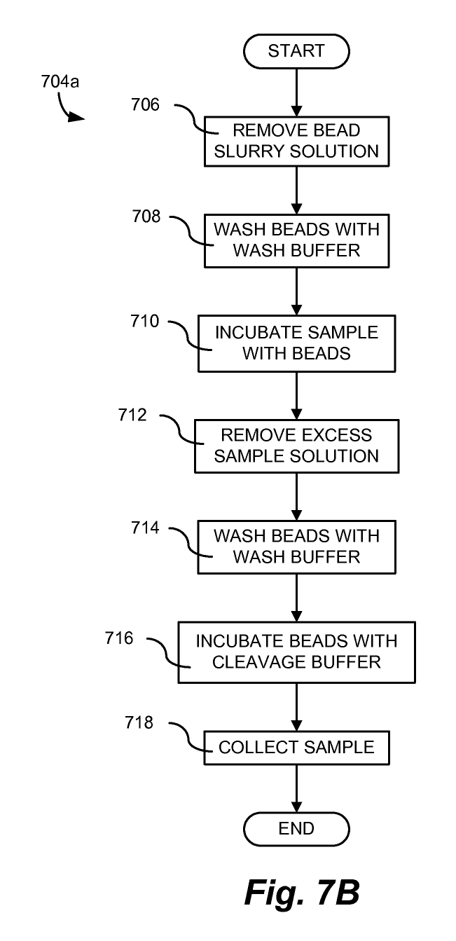

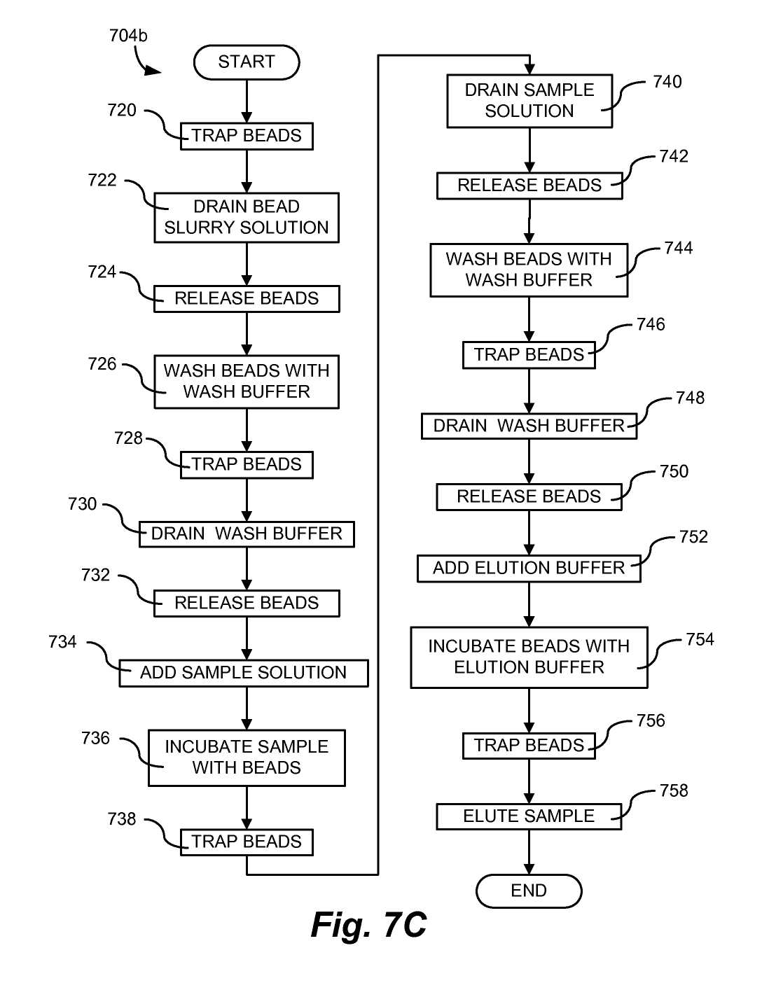

Reservoirs: The present device typically includes at least one reservoir for containing a fluid, and performing any functions on the fluid. For example, flexible cartridge fluid analysis devices of the invention have one or more fluid reservoirs that are physically manipulated by external forces, for example pumping or valving via a piston, as part of an analysis protocol that, for example, is used to isolate a target species within the fluid sample. An exemplary process flow is described in relation to FIGS. 7A-C below.

A portion of the internal pouch body itself may function as such reservoir.

The pouch body may be compartmentalized, forming a fluidic circuit, such that when the compartments are connected, they are in fluidic communication (that is, fluids may flow between or among reservoirs or chambers, and herein the terms are used synonymously). Thus, the internal pouch body may itself comprise, consist of or consist essentially of a reservoir.

The internal pouch body may be further partitioned such that uncombined moieties may be separately contained, and later admixed upon initiation of fluidic movement. As an example, a fluidic pouch device, for example as depicted in FIG. 1, may have additionally attached thereto further reservoirs fluidically linked with the central reservoir comprising the fluidic circuit.

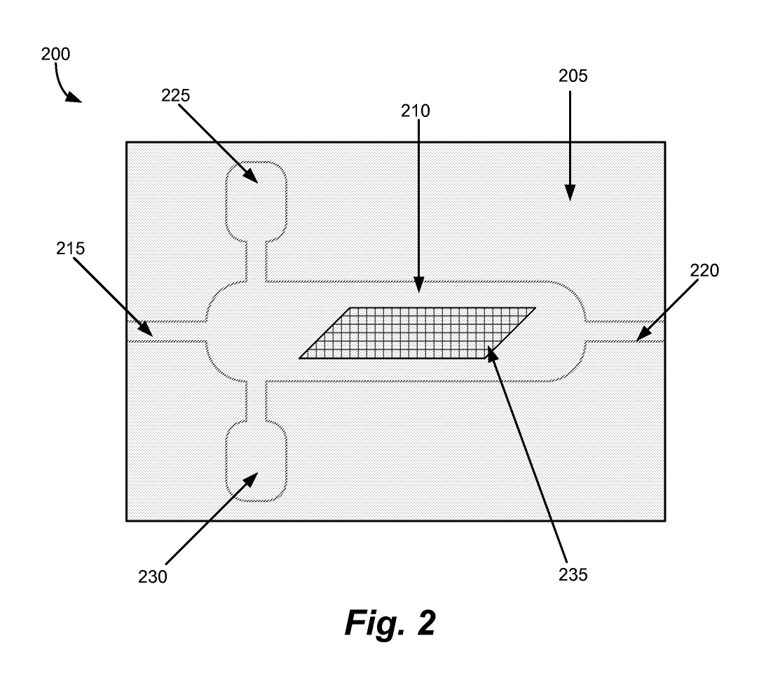

Referring to FIG. 2, the pouch may comprise a fluidic circuit containing reservoirs fluidically connected. These additional reservoirs may be partitioned such that the fluids are admixed upon applying external force. The flexible pouch has a unitary body, 205, with fluidic circuitry. The flexible pouch is configured with one input port, 215, for a sample and one outflow port, 220. Fluidically connected to the input port is a reservoir (or chamber), 210, central to the flexible pouch device. Also connected to central reservoir 210 are two additional reservoirs, 225 and 230, in fluid communication with the central reservoir via fluid channels. The central reservoir is illustrated here with a ferromagnetic trapping station, 235, for use with magnetophoretic applications (such as magnetophoretic cell sorting).

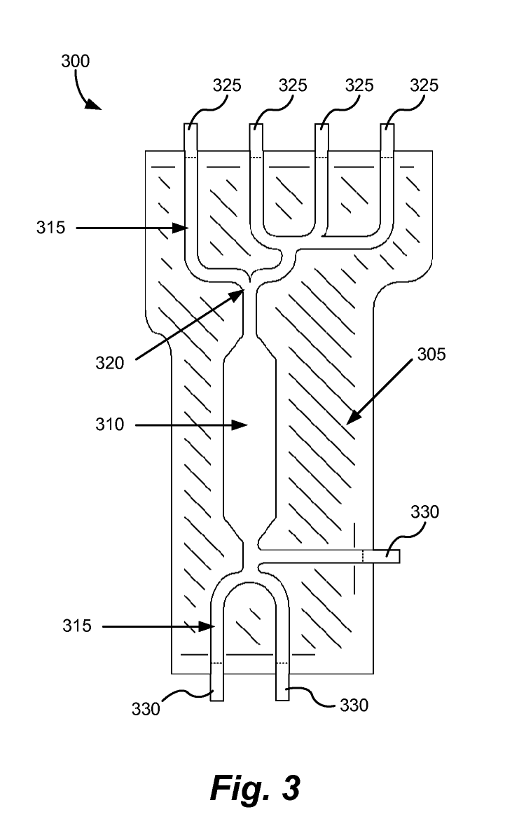

FIG. 3 is a block diagram of a flexible polymer pouch fluid analysis device, 300, showing more complex fluidic circuitry. Device 300 has a unitary body, 305, like the pouch devices described in relation to FIGS. 1 and 2. In this example, the fluidic circuitry includes inlet ports, 325, fluid communication conduits, 315, confluences, 320, in the conduits, and exit ports, 330. Further circuitry and function is derived in device 300 via external manipulation, for example, valving which is supplied by locally deforming device 300. For example, a fluid communication conduit, 315, can be pinched off thereby isolating one port from a reservoir, for example, stopping flow to the reservoir or partially closing off to meter a reagent into a reservoir. Such external manipulation can be, for example, by manual manipulation or by an apparatus designed to register with and manipulate device 300 as described.

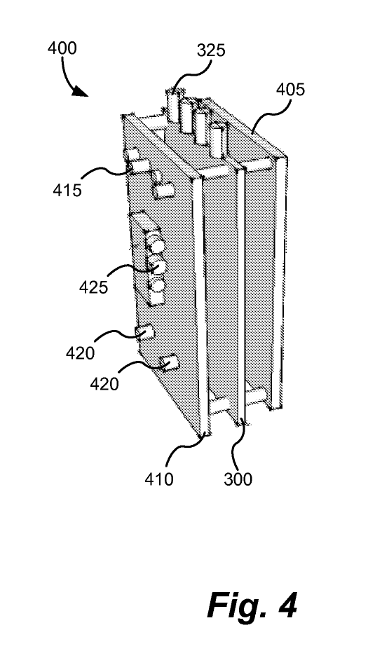

FIG. 4 is a block diagram of a clamshell, 400, (nest holder) system prophetically containing, for example, flexible pouch fluid analysis device 300 as described in relation to FIG. 3. In this example, inlet ports 325 of device 300 are seen emanating from the top of clamshell 400. Clamshell 400 has a back plate 405 and a front plate 410, between which is registered device 300. One or more fluids are added via inlets 325, and once in device 300, are manipulated via external forces applied to device 300 via clamshell 400. For example, clamshell 400 includes pinch valves (pistons) 415 and 420 and cam driven pumps 425 which apply forces to device 300 in order to manipulate liquids therein, for example, valving, mixing, etc. Clamshell 400 can also have magnetic, acoustic or other sources to manipulate particles in the fluids inside device 300 susceptible to such forces. When flexible pouch devices are used with a clamshell, the clamshell may also include components for suspending the pouch device for proper registration with manipulative components of the clamshell. When a flexible cartridges are used, the cartridges generally are rigid enough to be self-supporting, the clamshell need only provide enough space to accommodate registration of the flexible cartridge device. More detailed aspects of clamshell apparatus of the invention are described below.

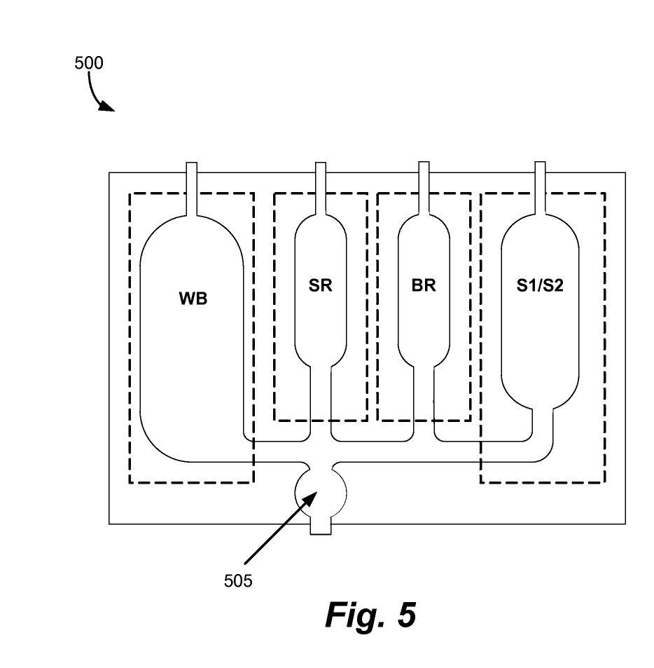

FIG. 5 is a schematic illustration of a fluidic circuitry configuration, 500, for the present flexible pouch or cartridge devices. Circle 505, where the channels connect, indicates a mixing chamber and/or magnetic trapping area. Dotted lines indicate the areas that may be subject to tensile pressure for fluidic movement. In this illustration, “WB” indicates “wash buffer”, S1/S2 indicates sample 1/sample 2, and “BR” indicates bead release buffer. In this example reservoirs have a distinct purpose related to carrying out one or more isolation or characterization protocols with the flexible pouch device.

Reservoirs may serve a functional purpose, and comprise additional structural elements to effect such purpose. For example, where cultured cells are desired, one may have suitable cell culture apparatus integrated with the reservoir, such as (but not limited to) aeration devices, mixers, or temperature controls. These devices may form a part of the present flexible pouch device, or may be part of related processing instrumentation wherein their device integration is temporary (when the flexible pouch is operably connected to the instrumentation).

Reservoirs may be adapted to operate within a system for fulfilling a function.

Another advantage of employing thin polymeric materials in pouch and cartridge devices of the invention is that manipulation via external forces is more flexible and accessible than in rigid devices. For example, in order to perform magnetophoretic separation in a flexible pouch, one can bring an external magnet closer to the sample than in a rigid device because of the thin flexible material used for the pouch. In another example, the pouch can be manipulated such that, for example, magnetized and localized material is selectively isolated by, for example, folding the pouch in half to isolate the localized material in a separate compartment or by heat sealing the localized material in a separate compartment. Once isolated to a separate compartment, the material can be removed with the compartment intact, and/or the compartment punctured to transfer the material for further processing.

Separating particles from a suspension may be particularly advantageously performed in the present flexible pouch device. For example, where magnetophoretic separation of a particular moiety from a complex mix is desired, one may have suitable magnetophoretic trapping structures in place (such as a ferromagnetic structure). With the present flexible pouch devices, such trapping structures (for magnetophoretic or other types of trapping structures) may or may not be adhered to the internal wall of the pouch. This may prevent non-specific binding. In substantial contrast to rigid microfluidic devices, for example, the present magnetophoretic trapping structure may be suspended in liquid, and operably controlled by magnetic controllable forces external to the device (for example).

Another advantage of magnetophoretic separation in the pouch or cartridge of the invention is the ability to bring the external magnets closer to the sample than in a rigid device by way of using thin material, manipulate the pouch such that magnetized material is selectively captured in certain areas, for example, folding the pouch in half to create layers of captured material.

Device Functional Components: One may configure or adapt the present devices for filling and measuring fixed volumes, or for continuous flow. One may configure or adapt the present devices for multiplexed functions (such as cell lysis and protein isolation). The present device may be configured for a single or multi-step process or assay, and may be configured for reagent storage. One may include filtration configurations or adaptations.

For example, the present flexible pouch device comprising a fluidic circuit may further comprise a structure for trapping target moieties, as a form of particle sorting, for example. For example, one may include a ferromagnetic grid for magnetophoretic particle sorting, to act as a “trapping station” for magnetically bound target species.

Magnetophoretic microsystems are finding increasing use in biotechnology and biomedicine for applications such as bioseparation and immuno-assays. These microfluidic systems typically contain embedded elements that produce a magnetic field distribution within a microchannel. This applied field gives rise to a magnetic force, which acts to manipulate or trap magnetic micro or nano-particles as they flow through the channel. Magnetophoretic microsystems are well suited for bioapplications because they enable fast reaction times, the analysis and monitoring of small samples, and integration with analytical instrumentation.

### Integration:

Integration of functional elements may be accomplished any number of ways. In general, one may fluidically connect access ports and reservoirs in all combinations via flow channels. Flow channels may be adapted for the kind of flow so desired, and may be of any dimensions that permit desired fluidic movement.

Flow control structural elements may be selected from a wide variety. These can be valves, porous membranes, mixers, pumps, porous membranes, and other traditional flow control structural elements. One may have traditional flexible pouch closures, such as zip locks, adhesives, heat seals, and other sealing types frequently used in the product packaging field. Alternatively, one may release material that solidifies in situ to block flow.

Moreover, if the present flexible pouch material is appropriately selected, the flow control may be temporary. If the material is elastic, retaining shape after deforming, one may provide tensile pressure to direct flow, or even define reservoir or flow channels. One may, for example, simply clamp a flow channel, or use magnets tightly bound on either side of the device to define a reservoir.

### Material:

Composition: The choice of pouch fabrication material is non-limiting, and is influenced by factors such as the product contained in the pouch, the shape of the pouch, or the anticipated use of the pouch. For ease in commercial manufacture, the flexible pouch is practicably formed from a roll of material comprised of flexible material, and typically will include a polymer. The material may be comprised of laminate layers, and include metallic, ceramic, glass or other components, as desired. The material may include co-extruded polymers to form a laminate having, for example, barrier layers to exclude oxygen, light or other external factors. Typically, for use with live biomaterials, such as cells in culture, the material will be biocompatible so as not to have a deleterious effect on cells in culture. In one embodiment, the material may have agents incorporated into it, for example, antibacterial agents, grids of ferromagnetic materials such as nickel and nickel alloys (for magnetic separation), antibodies and the like. One may choose to have a particularly porous material allowing for oxygen flow yet preventing potentially contaminating organisms. The present flexible pouch devices may comprise different materials in different geographic locations, such as a metallic material where current conductivity is desired, an optically clear material where visibility of the internal contents is desired, and an opaque material permitting protection of (for example) light sensitive materials. The material may be suitable for storage under various conditions, such as freezing, heating (as for example autoclaving), or exposure to various gasses (for example, ethylene oxide for sterilization). The material may be particularly chosen for adaptation to surrounding environments, such as salt water or petrochemical exposure. The material may be of a grade suitable for governmental compliance regimes, for example, food, medicament, device, etc.

One may choose to have a pre-printed outer layer, for example, and a portion not-so preprinted i.e. translucent, in order to view the contents contained therein. The clear portion could be in a gusset or insert. An outer layer of material may include preprinted information.

Physical properties: Strength, flexibility, and plasticity and elasticity are all important considerations for choosing material. Generally, for fluidic flow handling and control, the material should have physical properties permitting the external force contemplated for controlling the internal fluid flow. For example, if rollers are used to roll fluid from one reservoir to another, the material should be chosen in contemplation of the strength needed. The material should also be sufficiently flexible such that it will not crack under such force (or other conditions) used. Moreover, one may choose to have an external frame of relatively rigid material, so that the pouch itself may be flexible, yet the device is adapted via use of a rigid frame for suitable automated instrumentation. One may desire a “clam shell” configuration. Other types of external features may be included, depending on the contemplated applications.

A variety of polymers may be used including but not limited to polyethylene, polypropylene, polystyrene, polybutylene, polyvinylchloride, polytetrafluoroethylene (PTFE, teflon), polycarbonate, polyethylene terephthalate (PET), polyester, polyamide, polymethylmethacrylate (PMMA), polyetheretherketone (PEEK, polyetherketone), nylon and fiber reinforced plastics or resins. In one embodiment, the material includes a biodegradable polymer, for example, plastarch, polylactic acid and the like. The thickness of the flexible pouch and/or cartridge can vary from between about 0.001 mm to about 3 mm, in another embodiment between about 0.005 mm and about 2 mm, in yet another embodiment between about 0.005 mm and about 1 mm, in another embodiment between about 0.01 and about 0.5 mm. Using polyethylene, for example, thickness can range from 0.005 mm (similar thickness to common grocery bag) to 2 mm.

The materials may be UV resistant, rust resistant, scratch resistant, tarnish resistant and/or sterilizable. The materials may be co-extruded, multi-layer and the like.

A typical laminate material structure includes at least one layer of virgin polyethylene terephthalate (PET), at least one layer of aluminum foil and another layer such as EVOH, PET, polyethylene or nylon or the like. Another type of laminate material structure may also include a metalized foil paper layer laminated to a cast polypropylene layer and another layer of PET, polyethylene or EVOH. There may be a fourth layer of nylon. Similarly, the laminate structure may include a cast polypropylene (CPP) layer, a polyethylene (PET) layer, a foil (AL) layer, a nylon (ONO) layer and another CPP layer. Another structure is the use of nylon, foil, nylon and cast polypropylene (ONO/AL/ONO/CPP) or CPP/NY/AL/CPP. Another example of a material structure is ONO/AL/COEX-ONO-LDPE. Other materials suitable will be apparent to one of skill in the art.

Storage considerations: The pouch body architecture may be altered depending on the materials used. For example, certain laminates may begin to “creep” after a period of storage (particularly with a filled pouch device). The material may include an extrusion layer to contain “creepage” or “stretch” of the film after filling due to carbonation expansion, if the product is carbonated. In addition, the selected material may be organoleptic compliant in order to avoid the transfer of odor contaminates to the pouch product contents, or contamination during the shelf life period of the product. Temperature, humidity, light, condition fluctuations, and other environmental storage factors may be considered. External functional additions include (but are not limited to) handles, hang holes, zipper locks, tear notches, perforations, and anti-slip ridges.

All or part of the present device may be biodegradable, such as using polymeric material that degrades to non-toxic constituent moieties in the presence of heat, sunlight, water, etc.

Inventory considerations further include product identification, such as bar coding, RFID, or other means to identify devices. The present devices may be further packaged with related reagents. For example, for use with biological reagents, one package the present device with suitable buffers, media, detectable labeling moieties, apparatus (such as syringes for fluid fill), or other items.

Wash or Carrier Fluids: The present devices may be configured or adapted for use with, for example, aqueous buffers (with or without detergents), alcohols (methanol, ethanol, isopropanol for example), organic solvents (hexane, fluorocarbons, aromatic for example), or a combination of any of the above.

Electrochemical/electro-active: The present devices may be include one or more printed circuit boards, interdigitated electrodes, sputter or screen printed electrodes, or capacitance arrays. For example, one may pre-prepare flexible circuit boards on polymeric material, and use that to manufacture the present pouch devices.

Operability with forces used for particle trapping or sorting: One of the most promising applications for the present device is particle sorting, and there are number of ways this can be done. A controllable force, such as magnetic, acoustic, electrophoretic, or optical force is used to move a responsive particle suspended in a fluid.

Devices of the present invention can include magnetic activated particle sorting (such as cell sorting). Practicably, this involves using magnetic beads to which a selective binding molecule is attached. When the selective binding molecule binds to the desired target, the magnet is thus so attached. The desired target can then be trapped or sorted using magnetic force, and optionally a ferro-magnetic trapping station. Thus, a ferromagnetic material can be embedded in the flexible bag material, such as a printed magnetic area or by incorporating ferromagnetic dust particles into a polymeric substance (in a particular area, for example). As indicated infra, a ferromagnetic screen can be suspended in the liquid sample to achieve magnetic activated particle sorting.

Other controllable forces as are available in the art such as acoustic, electrophoretic or other forces can be incorporated into devices of the invention. A skilled practitioner will appreciate the appropriate device configuration to accommodate the separation system.

Optical Detection: The present devices may be configured to permit optical detection. This has practical applicability, for example, if colorimetric, fluorescent, or luminescent detectable markers are desired. Other optical interfaces may include fiber optic, surface plasmon resonance, attenuated total reflection or other optic interfaces.

Other Features: The present devices may be sterilized (such as with ethylene oxide, considering the durability of the selected material to other sterilization techniques, such as autoclavability). There may be surface compatibility with cell culture requirements, proteins and/or molecule compatibility, and additional surface energy in materials or configurations so selected. The present device may be, for example, gas permeable. Internal coatings, such as silicon, to minimize non-specific binding to the surface can be used in devices of the invention.

Flexible Pouch Device Manufacturing Systems: For commercial practicability, the present devices can use manufacturing techniques available for current pouch packaging. Generally, a pouch packaging machine is loaded with one or more rolls of packaging material, such as plastic film or paper. The packaging material is joined together along a common peripheral edge to create an enclosed pouch or bag. A product is placed in between section of the packaging material as the pouch is being formed. Accordingly, the product becomes packaged within the pouch as the pouch is formed. The products can be solid, granular, liquid, cream or even gaseous. The various pouches are then cut apart to create the individually packaged products that are ready for sale.

The present invention includes multiple flexible pouch devices connected on a single sheet, as it is contemplated that a manufacturing apparatus such as this will be used for greatest commercial convenience. Relatedly, one may adapt “blister packaging” equipment or other equipment for such purposes.

Typically, heat staking or adhesives are used for flexible pouch sealing, and one may so select these methods.

The present flexible pouch manufacturing systems include apparatuses for fluid fill, assembly, separating, coding (such as bar coding), sterilizing, and packaging.

The present manufacturing systems include those in compliance with various governmental or industry regimes, including food and drug requirements (for example, FDA, EMEA), quality control organizations (for example, International Organization for Standardization), and other regimes set up to ensure quality for a particular purpose.

Flexible Pouch Device Instrumentation Systems: [Brian—Should some or all of this section refer to “cartridges” as well as “pouches”?] The present devices and methods may be adapted or configured to work in conjunction with host instruments or to meet system requirements. Adaptations or configurations include (but are not limited to) one or more for vacuum filling, automated control of pumps and valves, pressure flow, injection loop for sample loading, temperature control, electro-osmotic flow, positive displacement pumping, expected volumetric flow rate, centrifugal force processes, humidity control, and gas exchange control, for example.

Fluid flow may be controlled by automated instrumentation, although depending on the device, one may use manual control. In remote locations, particularly, one may use external pressure via hand, or hand-tool.

Embodiments of the present invention also relate to the apparatus, such computers and microcontrollers, for performing these operations. These apparatus and processes may be employed to, for example, control a clamshell apparatus as described herein to perform processes for isolation of a target species from a fluid sample using a flexible pouch and/or a flexible cartridge of the invention. The control apparatus of this invention may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform and/or control the required method and processes.

Although the present flexible pouch devices may have integrated ports through which pneumatic (air or other gas, for example inert gas) pressure is used to control fluid flow within the device, the present devices may find particular advantage by being sealed and using external tensile pressure for fluid flow. As to gas flow, reagents in gaseous form may be added to the flexible pouch or cartridge of the invention to carry out chemical transformations within one or more chambers of the pouch or cartridge. Thus gases can serve two purposes both as a pressure element and as a means of delivering a reagent if in gaseous form.

The tensile pressure may be applied only to one side of a pouch (when supported against a solid support, for example), or on multiple sides. It may be advantageous to provide tensile pressure to opposing sides of the present pouch device. One may apply pressure to opposing sides of the present device, more specifically, front and back, or, if suitably configured, side to side. For example, if one is in a remote location, one may “pinch” the present flexible pouch device between the thumb and forefinger (for example) to induce fluidic flow. One may use mechanical means, such as opposing magnets, clamps, or other means to partition off a desired portion of the device and effect fluidic movement. This is advantageous not only in cost-to-make, but also, particularly for unindustrialized areas, cost-to-use.

Apparatus for direct downward (normal to the device's surface at the point of application of the force) tensile force pressure, sweeping tensile force pressure, or rolling tensile force pressure, or other kinds of tensile force pressure application may be used. Piston-type devices may be automated to provide downward (normal to the device surface) pressure. For example, there can be two pistons to provide tensile pressure in an alternating pattern, for example, where the two pistons apply pressure alternatively to different areas of a reservoir. This may be useful for mixing by fluidic movement in response to the pressure. A more elaborate device, with particular timing elements, may apply such pressure in sequence to particular functional areas of the present flexible pouch devices, such that fluidic movement through the fluidic circuitry is accomplished in a predetermined fashion. In another embodiment, pneumatic pressure is used to create the tensile force, for example, to push or sweep against a volume of the flexible pouch device.

Rollers or “bar” type apparatus (such as a “windshield wiper” type of movement) operating in an automated fashion over selected portions of the present flexible pouch devices may be desirable, particularly where the flexible pouch material may be subject to tearing with undue stress. Herein, such force is referred to as “sweeping” tensile force, in reference to the sweeping movement.

For applying pressure to opposing sides, one may place the present flexible pouch device between opposing rollers (or bars, or combination, for example). The rollers (for example) may be only on a portion of the device, such as a particular compartment or reservoir (as described more fully below), or on a channel through which the fluid may flow. A variety of configurations and ways to apply such tensile pressure may be used.

Various types of rollers may be suitable, including (but not limited to) polyurethane rollers, natural or synthetic rubber, neoprene, silicone, and metallic. The rollers may be adapted from currently available cleaning rollers, conveyor rollers (forming roller beds), “dead shaft” or other rollers (having bearing around a shaft that is immovable), distribution rollers (for depositing, for example, material on the surface of the device, typically ink, but here, for example, ferromagnetic particles or other materials for particle separation, cylindrical roller, stringer rollers, “V” rollers, and web spreader rolls. if tensile pressure for controlling fluidic movement.

The amount of pressure for a desired amount/rate of flow may be calculable based on fluid dynamics considerations, including: compressible versus incompressible flow, viscous versus inviscid flow, steady versus unsteady flow, laminar versus turbulent flow, Newtonian versus non-Newtonian fluids, subsonic versus transonic, supersonic and hypersonic flows, non-relativistic versus relativistic flows, magnetohydrodynamics, and other approximations according to methods known in the art. One of ordinary skill in the art will consider fluid dynamics in view of the overall system, including the pouch and/or cartridge materials.

### Flexible Cartridges and Related Apparatus:

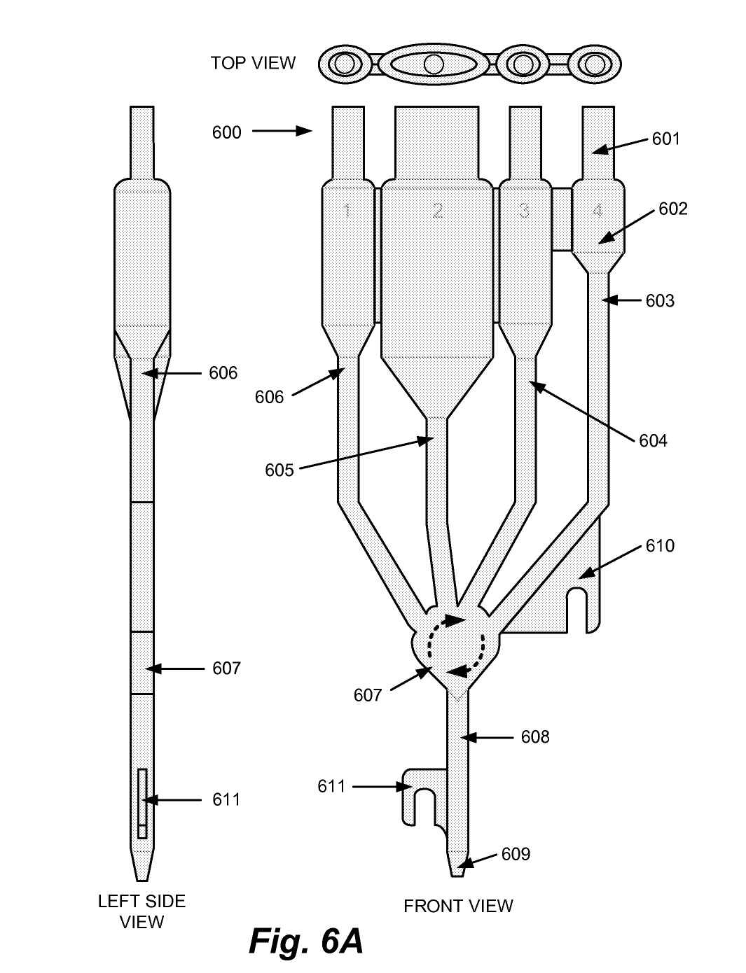

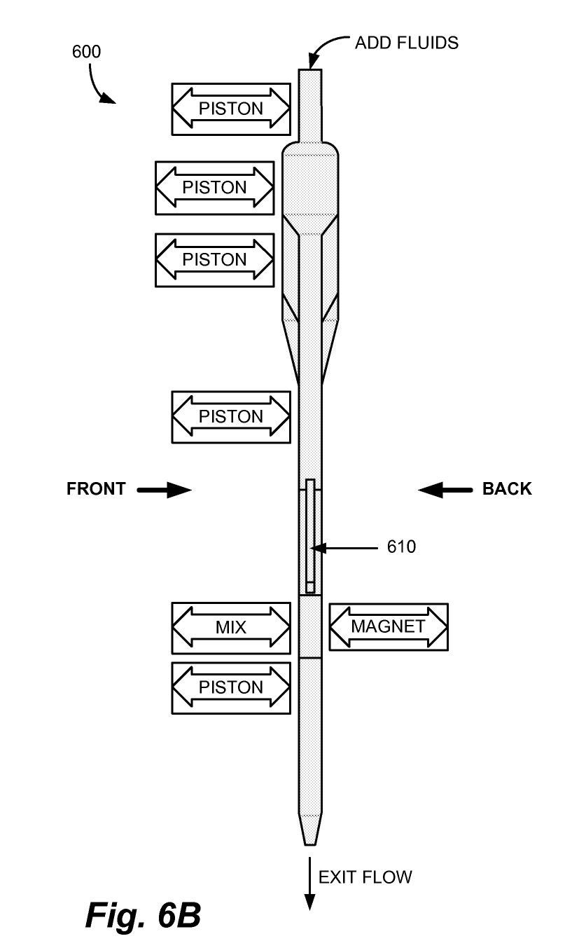

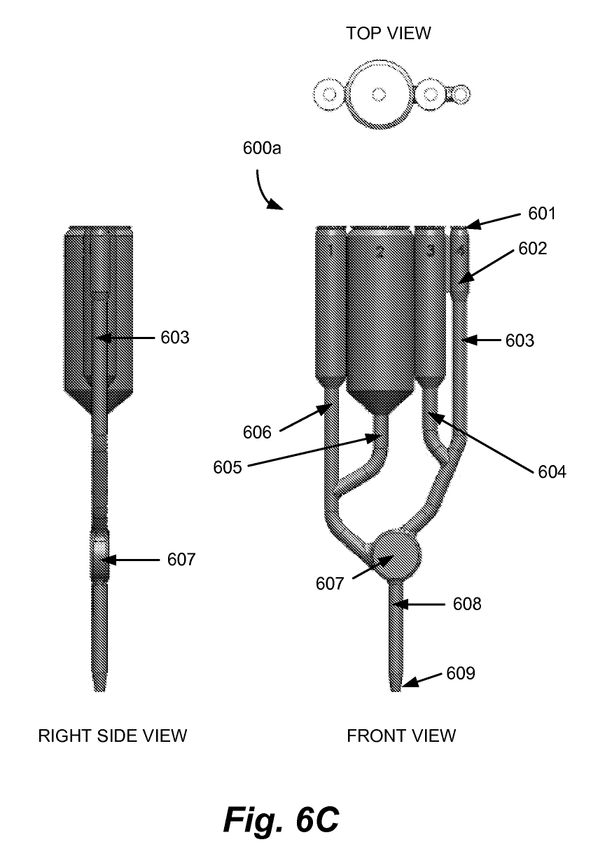

FIG. 6A, depicts a flexible cartridge fluid analysis device, 600, of the invention. Cartridge 600 is a single unit formed from, for example, blow molded polyethylene, polypropylene and the like. FIG. 6A depicts the top view, left side view and front view of cartridge 600. Referring to the front view, cartridge 600 has four access ports, for example port 601. Fluid sample, wash buffer, magnetic beads, and other reagents are loaded into cartridge 600 via the four access ports at the top of cartridge 600. The top view shows that the access ports in this example are oval in shape and each lead to a corresponding reservoir, labeled 1-4 in this example as part of the blow molding process. For example access port 601 is in fluid communication with reservoir 602 (labeled on the actual device as “4”). In this example, markings such as reservoir number, are formed on the device, for example, during blow molding. Thus, one embodiment is the flexible cartridge described herein with markings on the device that are formed as part of the blow molding process). Markings can include numbering, volume graduations and the like. In fluid communication with each reservoir is a fluid channel, for example, reservoir 602 is in fluid communication with fluid channel 603. The circular aperture of fluid channel 603 can be seen at the bottom of reservoir 602 from the top view. Each of the four reservoirs in this example is in fluid communication with a corresponding fluid channel, for example, channels 604, 605 and 606. Each of the reservoirs, 1-4 in this example, have an associated volume. In this example, reservoirs 1 and 3 have the same volume, while reservoir 2 has a larger volume than all the others and reservoir 4 has a relatively smaller volume than reservoirs 1-3. In a specific embodiment, the reservoirs have volumes of about 1 ml, 5 ml, 1 ml and ½ ml, for reservoirs 1-4, respectively.

Each of fluid channels 603-606 lead to a mixing chamber, 607. The dotted arrows on mixing chamber 607 indicate the general mixing area. Mixing can be achieved, for example, via an external magnet applying force to magnetic particles in a fluid sample, or for example, by pneumatic pressure applied to one or more of access apertures, like 601, which move fluid in, out and around inside mixing chamber 607. Mixing chamber 607 can be used to mix reagents with a fluid sample, and/or as a venue for separation of a target species from the fluid sample. In fluid communication with mixing chamber 607 is a fluid channel, 608. Fluid flows from mixing chamber 607, for example via gravity if device 600 is oriented vertically or via applied pneumatic or pumping pressure, to fluid channel 608 and exits device 600 via exit port 609.

As mentioned above, the term “cartridge” is meant in the conventional sense, that is, a container for, in this case, liquid made for ready insertion into an actuation instrument, a device or mechanism that manipulates the container, in this case for fluid handling and analysis. Flexible cartridge fluid analysis device 600 is intended to be used in conjunction with an apparatus that supplies valving, pumps, delivers fluids to access ports, collects fluids via port 609, applies external magnetic force and the like. In this example, device 600 includes registration ears, 610 and 611, for registering the device within an actuation instrument that holds the flexible cartridge (or pouch) device in operable position and provides multiple computer controlled external actuators for interacting with or providing fluidics components in the cartridge (or pouch). An example of an actuation instrument is a clamshell apparatus, for example, similar to that described in relation to FIG. 4. As seen in FIG. 4, the access ports of the flexible cartridge device are accessible to the user, or in another example, there is a “lid” that covers the access ports for delivery of fluids to each of the access ports as well as application of vacuum and/or pneumatic pressure to move fluids within the device and/or supply inert atmosphere if the device is used for air sensitive applications. Thus, the clamshell device is opened, cartridge 600 is inserted and properly registered via registration ears 610 and 611, the front plate is closed (“closing the clamshell”) and the lid then closed or applied atop one or more of the cartridge access ports. In one embodiment, not all of the access ports are covered, in another embodiment, the lid covers all the access ports for application to the access ports of fluids, pneumatic pressure and the like. For example, once registered in the clamshell, operations for isolating a target species from a fluid sample are carried out within the cartridge, and when complete, the clamshell is opened and the cartridge removed so that a new cartridge can be inserted for the next process run.

In one embodiment, the clamshell device of the invention is in modular format, so that more than one clamshell can be adjoined in a single machine for parallel processing. In one embodiment, there are up to 20 such modular clamshell devices in a single machine, in another embodiment there are up to 10 modular clamshells in a single machine, in yet another embodiment there are up to 6 modular clamshells in a single machine. Machines housing modular clamshells of the invention may be configured so that the individual clamshells are in a row, one or more rows back to back, in a radial format about a central axis and/or combinations thereof.

FIG. 6B shows the right side view of device 600. Fluids are introduced via access ports at the top of device 600. In one example, the clamshell device has actuated pistons that apply physical pressure to device 600 at various points to pump fluid, or pinch fluid channels to cut off fluid communication (i.e. valve) typically reversibly during processing of a fluid sample. Valving is typically, but not necessarily, performed relatively close to a reservoir so as to keep the majority of a fluid within the reservoir rather than residing in the fluid channel, for example pinching the channels at points as indicated by arrows 603-606 and 608 in FIG. 6A. An external magnetic field can be applied to trap magnetic particles and release them by withdrawing the magnet or cutting off an electromagnetic field, for example. External magnets can be manipulated, for example rotated, for mixing magnetic particles in a fluid sample within device 600. By combination of such forces, device 600 becomes highly adaptable to many isolation and/or analysis protocols.

Note that the “mix” arrow in FIG. 6B indicates that a fluid sample can be mixed in mixing chamber 607, for example, by one or more methods including moving the fluid sample back and forth between two areas of the device using fluidic channels (for example flowing from 606 through 607 and ending up in 603, then back through 606 and back into 607 for example via pneumatic pressure), mechanical agitation, physical pressing of the pouch or cartridge (similar to pinching with one's thumb and forefinger together), magnetic mixing (by alternating a magnetic field on either or both sides of the device thereby causing magnetized particles to move back and forth within the device) and acoustic mixing (for example dipping a portion of the pouch or cartridge device in an ultrasonic water bath).

FIG. 6C depicts various views of another flexible cartridge fluid analysis device, 600a, of the invention. Flexible cartridge 600a is very much like flexible cartridge 600, the components of cartridge 600a having the corresponding component numbers as those for cartridge 600. The overall length and width of cartridge 600a is about the same as cartridge 600, but it is thicker due to larger reservoir volumes. In this example, cartridge 600a has reservoirs having volumes of about 5 ml, 25 ml, 5 ml and 2 ml, for reservoirs 1-4, respectively. One difference between cartridge 600 and cartridge 600a is that cartridge 600a has fluid channels 603 and 604 form a confluence (for example a “Y” configuration) prior to fluid communication with mixing chamber 607. Similarly, fluid channels 605 and 606 also form a confluence prior to fluid communication with mixing chamber 607. While not wishing to be bound by theory, this alternative configuration is believed to provide superior routing of fluids and minimize sample loss at the fluidic channel junctions, e.g. because mixing chamber 607 now has only two fluid entry channels, whereas in flexible cartridge 600, for example, there were four entry points for fluid channels (603-606). Also, by joining fluid channels at one or more “Y” junctions, it can also aid in instrumentation design because, for example, mechanical elements such as valves can be placed further apart, thus allowing for less complicated components for fluid handling in apparatus of the invention.

FIG. 7A is a process flow, 700, in accord with methods of the invention where a flexible cartridge fluid analysis device is used in conjunction with a clamshell device as described above. First, a cartridge is inserted into the clamshell device, see 702. Then the isolation and/or analysis protocol is carried out inside the cartridge using the clamshell device as described generally above. In one example, magnetophoretic beads are used to, for example, isolate a target species from a fluid sample. In one embodiment, the beads are optionally collected from the cartridge after the isolation protocol, see 760. In another embodiment, the cartridge is optionally sealed, for example heat sealed, after the isolation protocol (and optional bead recovery) is complete, see 770. After all desired processing is complete, the cartridge is removed from the clamshell, see 1280, and the process 700 is complete.

FIG. 7B depicts a more detailed process flow, 704a, of an isolation protocol using magnetic beads, where the clamshell employs, among other things, an external magnetic field to trap and release beads. This process flow can be carried out, for example, in flexible device 600 (as described in relation to FIGS. 6A-B), using reagents such as wash buffer, cleavage buffer to cleave target off the magnetic beads where it was attached via for example an antibody specific for the target species, magnetic bead slurry, and fluid sample, each in a separate reservoir, 1-4. First, the slurry fluid is removed from the magnetic beads, see 706. Then the beads are washed with wash buffer, see 708. Then the sample is incubated with the magnetic beads so that the target species is selectively bound to the beads, for example with the aforementioned specific antibody, see 710. Then the excess sample fluid is removed, see 712. Then the beads are washed one or more times with wash buffer, see 714. Then the washed beads are incubated with a cleavage buffer to cleave the target species from the beads, see 716. Finally the sample is collected and the process is complete, see 718. As mentioned in relation to FIG. 7A, optionally the beads are recovered as well.

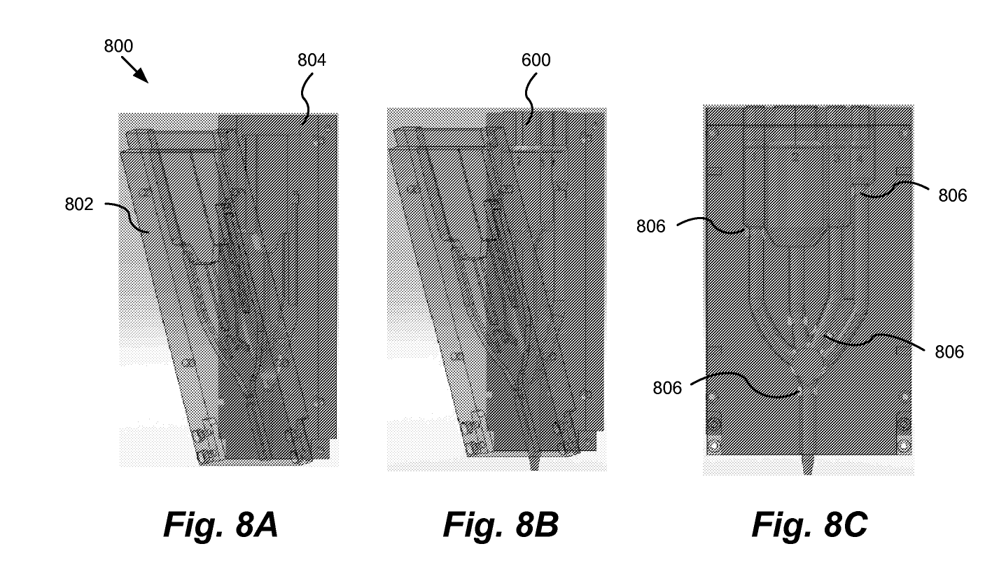

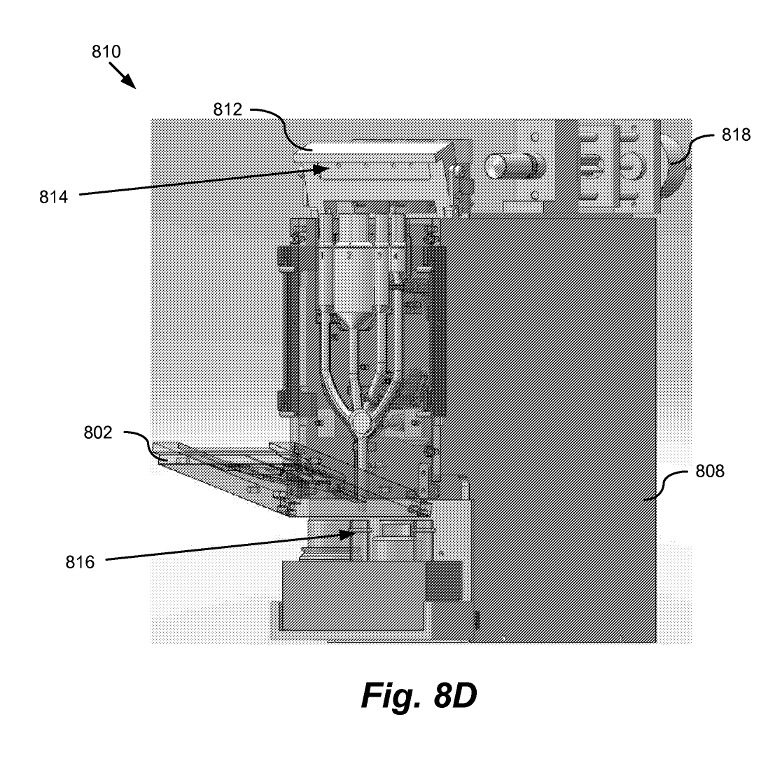

FIG. 7C depicts an even more detailed process flow, 704b, of an isolation protocol using magnetic beads, where the clamshell employs, among other things, an external magnetic field to trap and release beads. FIG. 8A is a schematic representation of a clamshell component, 800, that has a front plate 802 and a rear plate 804, that are used to support cartridge 600 in a clamshell apparatus. Note that each of the front plate and the rear plate are configured with recesses to accommodate cartridge 600 when inserted, see FIG. 8B. Once cartridge 600 is inserted, the front and rear plates are joined, see FIG. 8C. Also noted in FIG. 8C are valve actuators, 806. These valve actuators are employed when a particular section of cartridge 600 is meant to be isolated from another section via pinching off, for example, a fluid flow channel via one or more of these valve actuators. Note also in FIG. 8C, that the exit port of cartridge 600 protrudes out of the clamshell assembly, making collection of fluids from the cartridge more facile. FIG. 8D shows the modular clamshell apparatus, 810, that houses clamshell component 800 (front and rear plates). In FIG. 8D, cartridge 600 is depicted as nested in rear plate 804. Clamshell apparatus 810 has a body, 808, which includes motors, 818, pistons, magnetic field generators (permanent magnets drawn to a away from cartridge 600 or electromagnets that are turned on or off in proximity to cartridge 600, for example mixing area 607 (see FIG. 6A)) and the like to drive valve actuators as described in relation to FIG. 8C. Clamshell apparatus 810 also includes a top plate or lid, 812, which closes over cartridge 600 once the clamshell component is closed (front plate and rear plate adjoined). In this example, lid 812 includes pneumatic ports 814 which supply gas (for example air, inert gas, reagent gas and the like) pressure and/or vacuum or partial vacuum to one or more of the access ports in cartridge 600. Pneumatic valving serves to “close” each access port of cartridge 600 via back pressure which stops gas or fluid flow during certain operations as desired. Clamshell apparatus 810 also includes sample and/or waste vials, 816, or alternatively a waste or sample stream can run through a dedicated flow channel or line to a collection module or facility.

Referring again to FIG. 8D, the access ports of the flexible cartridge device 600 are accessible to the user prior to closing lid 812. A user can add reagents, sample and other fluids to each of the access ports of cartridge 600 and then close lid 812 for performing a process flow using the clamshell apparatus with cartridge 600. In another embodiment, the clamshell apparatus has a tray, for example as an integral part of lid 814, for preloading fluids for eventual introduction into access ports of cartridge 600 during process operations. Once cartridge 600 is registered in the clamshell, operations for isolating a target species from a fluid sample are carried out within the cartridge, and when complete, the clamshell is opened and the cartridge removed so that a new cartridge can be inserted for the next process run. Clamshell apparatus of the invention can be configured to manipulate flexible cartridge fluid analysis devices of the invention with varying configurations. One embodiment is a clamshell apparatus configured to manipulate (as described herein) flexible cartridge 600 or 600a.

Process flow 704b, of FIG. 7C can be carried out, for example, in flexible cartridge device 600 and utilizing clamshell device 800, using reagents such as wash buffer, cleavage buffer to cleave target off the magnetic beads where it was attached via for example an antibody specific for the target species, magnetic bead slurry, and fluid sample, each in a separate reservoir, 1-4. In this case, reservoir 1 is charged with fluid sample, reservoir 2 is charged with wash buffer, reservoir 3 is charged with cleavage buffer (or elution buffer as it's sometimes called), and reservoir 4 (601) is charged with magnetic bead slurry. In this example, the clamshell device of the invention has a tray, that can be preloaded with the aforementioned fluids and, once the lid is closed, the fluids are delivered to the corresponding access ports via the tray during process operations.

FIG. 7C will be described in detail along with FIGS. 9A-L, which depicted valving, magnetic and other operations performed on flexible cartridge 600 in order to carry out process flow 704b for isolating a target species from a fluid sample using magnetic beads. In FIGS. 9A-L, as depicted, a white filled rectangle indicates an open valve, that is, no pinching of the device at the location of the rectangle, and a black rectangle means a closed valve, that is, pressure is applied to close off the fluid channel at the position indicated by the rectangle. The applied pressure is reversible, thus allowing opening and closing of valves on the device. A white-filled circle indicates no applied magnetic field, magnet off, at the mixing chamber 607, while a back circle indicates an applied magnetic field at the mixing chamber in order to trap magnetic particles. Thus the valve below reservoir 1 is called the “sample valve,” the valve below reservoir 2 is called the “wash buffer valve,” the valve below reservoir 3 is called the “cleavage buffer valve,” the valve below reservoir 4 is called the “bead valve” and the valve below the mixing chamber is called the “outlet valve.”

Referring FIG. 9A, in conjunction with FIG. 7C, before any fluids are added to any of reservoirs 1-4, and after the cartridge is loaded into the clamshell and the tray is loaded with the respective fluids as described above, the access ports to reservoirs 1-3 are closed, the fluid channel below reservoir 4 is closed (bead valve), the magnetic field is applied to the mixing chamber and the fluid channel below the mixing chamber is closed (outlet valve). Magnetic bead slurry is then pipetted into reservoir 4.

Referring to FIG. 7C, the beads are first trapped, see 720. Specifically, referring to FIG. 9B, the bead valve is opened allowing the beads to flow into the mixing chamber, where they are trapped by the applied magnetic field. Sample, wash buffer and cleavage buffer are added to reservoirs 1-2 respectively, via opening the corresponding access ports, and closing the sample valve, wash buffer valve and cleavage valve. Then the outlet valve is opened, to drain the slurry fluid from the beads, see 722.

Referring to FIG. 9C, access ports 1-3 are closed along with the outlet valve. The magnetic field is removed so as to release the beads in the mixing chamber, see 724. The wash valve is opened and closed to allow a portion of wash buffer from reservoir 2 to enter the mixing chamber and suspend the beads. In this example, pneumatic pressure is applied and released one or more times via the access port of reservoir 4 in order to create an agitating action of the slurry of beads in wash buffer in the mixing station, see 726.

Referring to FIG. 9D, the magnetic field is then applied, see 728, at the mixing station and the outlet valve opened to release the portion of wash buffer used to wash the beads, see 730. Typically this waste stream is collected in a waste vial or a dedicated waste stream of the clamshell device.

Referring to FIG. 9E, the outlet valve is closed, the magnetic field is turned off, the access port to reservoir 1 is opened and the sample valve is opened to allow the sample to enter the mixing chamber with the beads, see 734, FIG. 7C. The sample is next incubated with the beads, see 736. In this example, pneumatic pressure is applied and released at the access port, for example, to reservoir 1 or 4 in order to agitate the sample and the beads together. In another embodiment, pneumatic pressure is applied alternatively to both access ports in order to create agitating action during incubation of the beads with the sample.

Referring to FIG. 9F, the magnetic field is applied to trap the beads, see also 738. The outlet valve is opened to drain the sample solution (with any unattached sample), see 740.

Referring to FIG. 9G, the outlet valve is closed and the magnetic field turned off, see 742. Wash buffer is added via opening and closing the wash buffer valve, and the beads (with attached sample) are washed using the agitation methods described above (note 9G is the same valving configuration as 9E), see 744. Referring to FIG. 9H, the magnetic field is turned on to trap the beads, see 746. Then the outlet valve is opened to release the wash buffer, see 748. This cycle of washing and releasing the wash buffer is repeated two or more times, in one example four times, until the beads sufficiently washed.

Referring to FIG. 9I, the outlet valve is closed and the magnetic field is released, see 750. The cleavage buffer valve (on reservoir 3) is opened and closed to allow cleavage buffer into the mixing chamber, see 752. The beads are then incubated in the mixing chamber with the cleavage buffer in order to cleave the target species from the beads, 754. As above, the incubation is typically, but not necessarily, concurrent with agitation as described using pneumatic pressure.

Referring to FIG. 9J, the magnetic field is applied in order to trap the beads, see 756. It is noteworthy that when trapping the magnetic beads, the agitating action applied, for example when incubating the beads, can be applied to ensure capture of a maximum amount of the beads—this is done prior to opening the outlet valve. Once the beads are trapped, the outlet valve is opened allowing the sample to elute to a target vial, see 758, and process flow 704b of FIG. 7C is complete.

Pneumatic pressure may be applied to any elution or draining process to aid moving fluid out of device 600, as there may be resistance to flow due to capillary action in the fluid channels (depending on the size of the device and channels). In one embodiment, device 600 is about 6 inches long, about 2 inches wide and about ¼ inch thick. The reservoirs in this example have volumes of about 1 ml, 5 ml, 1 ml and ½ ml, for reservoirs 1-4, respectively.

Referring to FIG. 9K, once the sample is collected, the outlet valve is closed, and the magnetic field is turned off. Wash buffer is added to the mixing region and the beads agitated in the wash buffer to ensure freedom of movement in the slurry, that is, to free beads that may be clinging to the sides of the mixing chamber. Then the outlet valve is opened and the beads are collected, see FIG. 9L. As mentioned with reference to FIG. 7A, the cartridge can be sealed, for example heat sealed, and then removed from the clamshell device for disposal or recycling, for example after autoclaving.

Other aspects of the invention will be apparent to the skilled practitioner from the description herein.

### Applications:

Because the present flexible pouch and flexible cartridge devices and related methods and systems essentially provide the function of other analytical, sorting and separation devices, it is widely applicable. Applications include biological fluid sample preparation and analysis, separation of rare molecules or cells, chemical library screening, point of care diagnostic in a clinical laboratory setting, environmental testing or monitoring, consumer products and food quality control aspects, for example. Biological fluids include amniotic fluid, aqueous humor, blood and blood plasma (and herein blood refers to the plasma component, unless otherwise expressly stated or indicated in context), cerumen (ear wax), Cowper's fluid, chime, interstitial fluid, lymph fluids, mammalian milk, mucus, pleural fluid, pus, saliva, sebum, semen, serum, sweat tears, urine, vaginal secretion, and vomit.

In particular aspects, the present devices may be configured or adapted for cell lysis, bead-based displacement assays, perfusion, filtration, sample preparation, chemotaxis, whole blood separation, protein purification, molecular separation and/or purification, and a variety of other biological and chemical materials and processes.

The present devices, methods, manufacturing systems and instrumentation systems may individually or in any combination be configured for cell selection and optionally culturing in situ.

One may select particular stem cells, for example, from blood, marrow, umbilical cord or other sources, and optionally, culture cells so selected in situ. One may use moieties selective for stem cells, such as CD34+ selective binding molecules (meaning molecules that selectively, but perhaps not specifically, bind CD34 protein, such as antibodies or aptamers). Such selective binding molecule may be connected to a moiety suitable for selection within the present device, such as a magnetophoretic bead, an acoustic bead, or other moiety capable of capturing the molecule (and cell) so selected.

One may select particular circulating tumor cells, for example, from blood of a patient being monitored for a cell proliferation disorder (such as cancer).

Prefilled “Kit in a pouch”: Because of the ease in manufacture and use, it is contemplated that one aspect of the present invention is a flexible pouch device prefilled with reagents useful for a particular purpose. For example, devices may be prefilled with reagents useful for biological sample preparation. This “kit in a pouch” aspect may be adapted for a variety of end users.

Such “kit in a pouch” aspects may include a variety of reagents and may be adapted for a variety of fields, such as biological fluid sample preparation and analysis, separation of rare molecules or cells, chemical library screening, point of care diagnostic in a clinical laboratory setting, environmental testing or monitoring, consumer products and food quality control aspects, for example. The present invention includes single or a plurality of prefilled devices suitable for such uses, and, as disclosed more fully herein, large numbers of the present flexible pouch devices may be rapidly prepared from sheets of flexible material. Configurations are non-limiting, but should be considered along with related instrumentation and methods.

The reagents may be disposed within the pouch device for ease of use, such as (but not limited to) in particular reservoirs in predetermined amounts. For example, a substantially purified protein preparation may be obtained by culturing cells so expressing the desired protein. The subject reservoir may be so adapted to culturing the cells, and have access ports with appropriate reagents in fluidic communication under controlled conditions.

The present flexible pouches may have reservoirs prefilled with suitable reagents. Reagents include buffers for lysing cells, washing cells, and removing beads selectively bound to a moiety. Additional reagents include selective binding molecules, such as antibodies, aptamers, and other molecules that selectively (although not necessarily specifically) bind a target molecule. Further reagents include various moieties allowing capture of the selected molecule, such as magnetic beads, acoustic beads and other beads providing that function.

The present invention further includes prefilled nucleic acids such as primers suitable for selecting particular nucleic acids from a complex mix. For example, the present device may be used to screen genomic DNA, and amplify selected sequences using polymerase chain reaction, within the device itself.

Additional processing modules or chambers may be added “upstream” or “downstream”. For example, if one uses the present flexible pouch containing fluidic circuitry for protein expression from cells in culture, one may have additional modules for derivatizing the protein so expressed, such as a pegylation module in which one may derivatize the subject protein with polyethylene glycol (or other polymer or other substance). One may so prepare post-expression modification fluidic circuitry, such as providing reservoirs with the desired polymeric substance (or other substance) for derivatization and reaction reagents.

Various sorting or detection modalities may be used. For example, one may use beads (suitable for magnetophoretic, or acoustic separation, for example) to which a selective binding molecule is attached. The selective binding molecule may not be specific for a particular target, but it binds selectively, rather than non-specifically or randomly. A skilled practitioner will be able to ascertain the degree of selectivity or specificity to be applied.

Selective binding molecules may be selected from among various antibodies or permutations (peptibodies, humanized, foreshortened, mimetics, and others available in the art), aptamers (which may be DNA, RNA, or various protein forms, and may be further modified with additional functional moieties, such as enzymatic or colorimetric moieties), or may be particular to a particular biological system. Proteins may be expressed with particular “tags” such as a “His-tag”, and a skilled practitioner will determine appropriate kinds of selective binding molecules or detectable labels are suitable.

Various portions of fluidic circuitry can be used for holding reagents so as to function in a process completed in the fluidic circuit. Reservoirs, such as those described for FIG. 2 may be prefilled, depending on the application.

Biological fluids: As indicated above, the present flexible pouch and cartridge devices may be configured or adapted for culturing, purifying or isolating components of, or analyzing a variety of biological materials. The present devices may be configured to perform one of more functions within the same device.

Cells and cell cultures may contain one or more stem cells, bacteria, human cells, bio-film materials, mammalian cells, yeasts, algae, primary tumor cells, immortalize cell lines, tissue or organ cultures, unicellular or multi-cellular organisms (considering the size and device configuration), molds and other organisms.

As such, the present device may not only have fluidic circuitry adapted for cell sorting (i.e., the stem cells), but also for expanding populations of stem cells. Additional characteristics of a bioreactor for stem cell growth may be used, such as media, oxygen, temperature, media replacement, and other characteristics.

The present device may be configured for sorting suitable stem cells from blood, and further culturing the stem cells for expansion. One may optionally include selected media, growth factors, and other materials to be pre-filled on a present pouch device. For example, one may use hematopoietic stem cell selective reagents, such as antibodies or aptamers. Such reagents may selectively bind to CD34+ stem cells. One may use a variety of techniques for isolating the CD34+ stem cells with a selectable reagent, such as magnetophoresis, where CD34+ stem cells are selectively attached to magnetic particles, which are then subject to a magnetic field. The magnetic particles, bound and unbound to CD34+ cells, are then held in place, and other material is washed away. A bead release reagent as commercially available, is then applied, and the bound cells are released. While the beads are captured by the magnetic force, the stem cells may be separated in a fluidic supernatant. These stem cells may then be further cultured and expanded in situ, or removing them for expansion in a different device.

The present devices may be used for tissue regeneration. For example, the present device may be configured with biocompatible scaffolding and suitable reagents for growing tissues ex vivo. Where the pouch device is used for stem cell expansion, reagents may be used to differentiate stem cells into different types of tissue-related cells. The present device may thus include a biocompatible scaffold or other support framework for cellular differentiation into tissue.

One may further culture liver or other organ tissues, for transplant, based on cells originally isolated and grown in situ in the present flexible pouch device. As the present device may be configured for stem cell selection and in situ expansion, one may further configure the device, including pre-filled reagents, for various applications involving stem cell differentiation.

If one seeks to implant the tissue, the pouch device itself may be made of biocompatible material so that the stem cell-grown tissue (or population of cells on a scaffold) may be applied or implanted directly into a recipient, such as a person. For example, if one desires to regrow cornea, the present pouch device may be so configured as described and including a portion of pouch material suitable for a corneal transplant (using the tissue so grown).

Research tool: The present flexible pouch and cartridge devices have broad use in scientific research, including but not limited to screening molecular libraries. For example, one may use the present device for screening aptamer libraries by preparing a purified and isolated protein on the present device, and then exposing the protein to an aptamer library, within a single device. (The term “aptamer” being used herein in its broadest sense to denote oligonucleic acid or peptide molecules that bind to a specific target molecule, and related synthetic molecules, such as mimetics)

Bio/Chemical Monitoring, Synthesis or Analysis: The present invention may be configured or adapted for a variety of biological or chemical monitoring, synthesis or analysis purposes, such as, for example, chemical threat monitoring, nucleic acid analysis (and amplification using, for example, polymerase chain reaction), continuous monitoring of particular conditions, such as closed environmental monitoring, personalized genomics and diagnosis, chemical synthesis, such as synthesis of aptameric therapeutics contained within viral coats or other nanocages suitable for delivery into a physiologic environment, or other chemical syntheses. For home use, for example, in monitoring swimming pool or drinking water quality, one may include pH indicator, metal indicators, or other indicators of water quality.

Other uses: The present devices may be configured or adapted for production process control, such as bioreactor monitoring in biopharmaceutical production processes, or for the food industry. The present devices may be adapted or configured for fluid control and analysis, gas control and analysis. For example, by adapting the present devices for continuous flow, one may monitor the rate at which cells are sorted. The present devices may be configured for production quality control, such as for supply chain monitoring.

### EMBODIMENTS

In accord with the description herein, one embodiment is a flexible pouch device including a fluidic circuit, the fluidic circuit optionally adapted for microfluidic flow. In one embodiment, the fluidic circuit includes a plurality of reservoirs fluidically connected. In another embodiment, the flexible pouch includes at least one fluidic circuit including at least one reservoir in fluid communication with another reservoir. Embodiments include the flexible pouch device as described and instrumentation providing tensile pressure to effectuate fluidic movement within the flexible pouch.

Another embodiment is a process for producing a plurality of the flexible pouch devices as described above where the plurality is manufactured on (or from) one or more sheets of polymeric material. One embodiment is a process for manufacturing a plurality of flexible pouch devices from one or more sheets of polymeric material, the method including: (i) arranging the one or more sheets of polymeric material so that there is an overlapping region of the polymeric material; and (ii) applying opposing plates with premilled molds to the overlapping region in order to form at least one flexible pouch device of the plurality of flexible pouch devices; wherein each flexible pouch device comprises at least one fluidic circuit including at least one reservoir in fluid communication with another reservoir.

One embodiment is a flexible pouch device including a pouch body that includes a fluidic circuit, where the fluidic circuit includes at least one reservoir in fluid communication with another reservoir, where the fluidic circuit is optionally adapted for microfluidic flow and at least one reservoir is prefilled with a reagent. In one embodiment, the flexible pouch includes a fluidic circuit including a plurality of reservoirs and a plurality of flow channels, adapted for effectuating fluidic flow by the use of tensile pressure. Another embodiment is a flexible pouch device as described above configured as in any of the figures described herein. One embodiment is the flexible pouch device as described, further adapted for particle separation selected from among magnetophoretic separation, acoustophoretic separation, and electrophoretic separation, or any combination thereof.

Another embodiment is a flexible pouch device adapted for sorting cells pre-labeled with a selectable binding agent from a cell suspension, the flexible pouch device including a pouch body including a fluidic circuit including at least one reservoir and at least one fluidic channel, the fluidic circuit adapted for flow of the cell suspension through the fluidic circuit; separation of the cells when pre-labeled with a selectable binding agent; collection of the separated cells within the fluidic circuit; and, optionally, configured for microfluidic flow. In one embodiment, the flexible pouch device is configured for sorting cells from bodily fluid sample. In one embodiment, the flexible pouch device is configured for sorting cells from a blood sample selected from among circulating tumor cells and hematopoietic stem cells. In one embodiment, the selectable binding agent is selected from an antibody and an aptamer. In another embodiment, the flexible pouch device of is configured to sort stem cells selectively labeled with an anti-CD34 selective binding agent. In one embodiment, the flexible pouch device is adapted for magnetophoretic, acoustophoretic, or electrophoretic, or any combination thereof. When adapted for magnetophoretic cell sorting, the flexible pouch includes a magnetically responsive trapping station.

Another embodiment is a flexible pouch device adapted for sorting particles pre-labeled with a selective binding agent from a particle suspension including: a pouch body including a fluidic circuit including at least one reservoir and at least one fluidic channel, the fluidic circuit adapted for flow of the particle suspension through the fluidic circuit; separation of the particles when pre-labeled with a selective binding agent; collection of the separated particles within the fluidic circuit; and, optionally, configured for microfluidic flow. In one embodiment, the flexible pouch device is configured for sorting particles from biological fluid sample, for example a blood sample. Examples of the selectable binding agent are an antibody and an aptamer. In one embodiment, the flexible pouch device is adapted for sorting stem cells selectably labeled with an anti-CD34 selective binding agent. In another embodiment, the flexible pouch device is configured for magnetophoretic, acoustophoretic, or electrophoretic, or any combination thereof. In one embodiment, the flexible pouch device is adapted for magnetophoretic particle sorting further including a magnetically responsive trapping station. In one embodiment, the flexible pouch device is configured to sort particles where the particles are indicative of condition selected from among a disease state, a drug concentration, and an infection.

One embodiment is a flexible pouch device adapted for screening a selective binding agent library in suspension against a target moiety including: a pouch body including a fluidic circuit including at least one reservoir and at least one fluidic channel, the fluidic circuit adapted for flow of the selective binding agent library in suspension through the fluidic circuit; binding of the selective binding agent members to a target moiety; sorting of the selective binding agents so bound to a target moiety within the fluidic circuit; and, optionally, configured for microfluidic flow. In one embodiment, the selective binding agent library is an aptamer library. In one embodiment, the target moiety is protein, in a more particular embodiment, the protein is a cell surface marker. In one embodiment, the sorting is magnetophoretic, acoustophoretic, or electrophoretic, or any combination thereof. In another embodiment, the flexible pouch device is adapted for magnetophoretic library screening further including a magnetically responsive trapping station.

Another embodiment is a flexible pouch device manufacturing system for manufacturing devices as described herein. In one embodiment, the flexible pouch device manufacturing system includes components for making individual flexible pouch devices on a single sheet of polymeric material or blow molded as a single unitary body.

Another embodiment is a flexible pouch device instrumentation system for operating a flexible pouch device as described herein. In one embodiment, the flexible pouch device instrumentation system of includes automated instrumentation for effecting fluidic flow using a tensile force. In one embodiment, the tensile force can includes at least one of a direct downward force, a rolling force, and a sweeping force. In one embodiment, the automated instrumentation includes rollers adapted for controlling fluidic movement within the fluidic circuit.

Another embodiment is a flexible cartridge for fluid analysis as configured as in any of the figures described herein, particularly FIGS. 6A-C. Another embodiment is a flexible cartridge for fluid analysis, including a unitary body made of a plastic material, the body including: (i) at least one reservoir in fluid communication with; (ii) a first fluid channel in fluid communication with; (iii) a mixing chamber, the mixing chamber in fluid communication with; (iv) a second fluid channel in fluid communication with; (v) an outlet for draining fluids from the mixing chamber. In one embodiment, the flexible cartridge further includes one or more registration tabs or ears for registering the flexible cartridge in an actuation instrumentation system for manipulating a fluid within the cartridge via application of one or more external forces to reversibly deform at least a portion of the cartridge. The external forces can be applied, for example, via a pump, a piston, a stepper motor, a pneumatic source and a roller. The actuation instrumentation system includes automated instrumentation for effecting fluidic flow using a tensile force. In one embodiment, the actuation instrumentation system is configured as a clamshell apparatus which holds the flexible cartridge during operation. In one embodiment, the flexible cartridge is adapted for manipulation of magnetophoretic particles in the fluid by application of an external magnetic field. In one embodiment, the flexible cartridge includes four reservoirs, each in fluid communication to the mixing chamber via the first, and a second, a third and a fourth fluid channel, respectively. The flexible cartridge can be made of one or more of materials, plastics work well. In one embodiment, the flexible cartridge (or pouch) is made of a material that includes at least one of polyethylene, polypropylene, polybutylene, polystyrene, polyvinylchloride, polytetrafluoroethylene, polycarbonate, polyethylene terephthalate, polyester, polyamide, polymethylmethacrylate, polyetheretherketone, nylon, fiber reinforced plastic, plastarch, and polylactic acid. In one embodiment, the flexible cartridge (or pouch) is made from a single material, for example, one of the aforementioned materials. In one embodiment, the flexible cartridge for fluid analysis as configured as in any of FIGS. 6A-6C is blow molded.

Thus another embodiment is a blow molded flexible cartridge for fluid analysis, including a unitary body made of a plastic material, the body including: (i) at least one reservoir in fluid communication with; (ii) a first fluid channel in fluid communication with; (iii) a mixing chamber, the mixing chamber in fluid communication with; (iv) a second fluid channel in fluid communication with; (v) an outlet for draining fluids from the mixing chamber. In one embodiment, the blow molded flexible cartridge further includes one or more registration tabs or ears for registering the flexible cartridge in an actuation instrumentation system for manipulating a fluid within the cartridge via application of one or more external forces to reversibly deform at least a portion of the cartridge. In one embodiment, the actuation instrumentation system includes automated instrumentation for effecting fluidic flow using at least one of a tensile force, a pneumatic force and a magnetic force, the automated instrumentation system configured as a clamshell apparatus which holds the flexible cartridge during operation. In one embodiment, the blow molded flexible cartridge includes four reservoirs, each in fluid communication to the mixing chamber via the first, and a second, a third and a fourth fluid channel, respectively. In one embodiment, the plastic material includes at least one of polyethylene, polypropylene, polybutylene, polystyrene, polyvinylchloride, polytetrafluoroethylene, polycarbonate, polyethylene terephthalate, polyester, polyamide, polymethylmethacrylate, polyetheretherketone, nylon, fiber reinforced plastic, plastarch, and polylactic acid. Another embodiment is a blow molded flexible cartridge for manipulation of a fluid sample, where the manipulation includes at least one of a cell separation, a protein purification and a molecular separation.

Another embodiment, is an automated actuation instrumentation system configured to manipulate the blow molded flexible cartridge described herein in order to carry out a procedure for isolation or identification of a target species in a fluid sample. In one embodiment, the automated actuation instrumentation system includes a clamshell assembly for supporting the blow molded flexible cartridge while performing the procedure.

Another embodiment is an apparatus for creating a fluidic circuit from a featureless bag, the apparatus including: (i) one or more molds and/or clamps which create the fluidic circuit upon engagement with the featureless bag; and (ii) one or more actuators for manipulating the fluidic circuit, the manipulation including valving and pumping a fluid sample within the fluidic circuit. In one embodiment, the apparatus further includes one or more external forces for manipulating a target species within the fluidic circuit, the one or more external forces including at least one of a magnetic force, an acoustic force, an electrophoretic force and an optical force.

### EXAMPLES

Set forth below are examples of making and using the present flexible pouches for fluid sample analysis.

Examples 1-3 are working examples. Example 1 is a working example describing the manufacture of pouch using a polyethylene bag. Example 2 is a working example of sample preparation using micromagnetic bead separation. Example 3 is a working example demonstrating the purification of protein from cells in culture using the present flexible pouch system comprising a flexible pouch and automated instrumentation providing tensile pressure.

Examples 4-10 are prophetic examples, illustrating various embodiments of the present invention.

### Example 1

### Manufacture of Microfluidic Pouch

Pouch Prototype: The prototype of the pouch was produced from 4 mil polyethylene bag (commercial product, Uline of Waukegan, Ill.) by fusing the pattern using Toman heat-staker and custom machined tool (end effector). The tool consists of two matching aluminum plates with machined opening and groves for tubing. The prototype pouch is shown in FIG. 1. The flexible pouch unit, 100, formed consisted of a 50 mm×20 mm rectangular body with a capsule-shaped volume, 105, in the middle of the body and running parallel to the length. At one end of the capsule-shaped volume was an inlet port, 110, and at the other end, an outlet port, 115. Thus, fluid flow into and out of the volume can be manipulated, for example, via pinching off the inlet or outlet and/or compressing the volume to move and/or mix fluid in the volume.

### Example 2

### Fluidic Sample Preparation Using Micromagnetic Bead Separation

Using 1 mL disposable syringe, the pouch from Example 1 was filled with water and blue food dye solution. The magnetic beads were subsequently injected into the pouch, creating a magnetic bead suspension. The pouch was sealed, and exposed to an external magnet. The magnetic beads localized within in the pouch at the location corresponding to the magnetic force (i.e., the external magnet).

When the pouch was placed over a 2-unit neodymium magnet stack. The beads instantaneously started to collect near the magnet.

A photograph taken approximately three seconds after the pouch containing the magnetic bead suspension was placed on the top of the 2-unit neodymium magnet stack all of the beads were captured in the surface directly over the magnet area. After the pouch was removed from the magnet, the beads remained in their localized mass at the location where the magnet came in contact with the pouch. Although not performed in this working example, one could then partition the localized magnetic particles by sealing off that portion of the present flexible pouch, for example by pinching off and heat sealing, thereby isolating the localized mass from the rest of the components in the pouch. Gentle pressure, for example by hand manipulation alternatively on either side of the capsule-shaped volume, re-suspended the particles as they were prior to exposure to the magnetic field.

### Example 3

### Isolation of Protein from Cells in Culture

The present working example demonstrates that the present flexible bag device can be used to purify and isolate protein from a cell culture expressing the protein in situ. Protein purification using a microfuge tube vs. using a flexible pouch device of the present invention was compared. Results, as visualized in a gel electrophoresis experiment, show that the flexible pouch was equally effective as the microfuge tube.

Cell pellet preparation and labeling with magnetic beads: One frozen pellet of E. coli expressing 27 kDa Glutathione-S transferase (“GST”) was thawed, lysed using 1 ml detergent (BPER-II), and exposed to washed magnetic beads (200 uL), via mixing using air pressure created with a pipette (without creating bubbles).

Two 500 μl samples were taken, one to be used in a microfuge tube, and the other for the present flexible pouch device. The aliquot in the microfuge tube was sealed in the tube, and placed in a lab rotator at room temperature for 40 minutes.

The other 500 ul aliquot was placed in the flexible pouch device. The flexible pouch device was configured using two devices as illustrated FIG. 1, end to end, such that two reservoirs were fluidically connected via a single flow channel. The aliquot was deposited within one of the reservoirs and sealed, and placed in a mixer for 40 minutes. The mixer presented two pneumatic pistons providing tensile pressure gently alternated in pumping motion in each reservoir, that is, pumping the fluid between the reservoirs for thorough mixing.

Thus, the incubation with beads was performed inside the flexible pouch (as well as the microfuge tube).

The microfuge tube and the flexible pouch device, each containing the 500 μl aliquot labeled with magnetic beads, were each placed against a magnet, and waste material was removed (for example, the material not so bound to the magnet). That material from the flexible pouch was saved, and a portion was run on a gel. Magnetic beads in the tube and pouch were washed 3×500 μl in wash buffer, thereby removing excess non-target protein. The magnetic beads were resuspended twice in 100 μL elution buffer, and agitated until resuspended which has the effect of releasing the target protein from the beads. Again, each of the tube and the flexible pouch was placed against a magnet, and the supernatant was drawn off (thereby leaving the magnetic beads in the magnetic field). The supernatant from each was prepared for gel electrophoresis.

Both samples from the lanes labeled “tube” and “bag” (referring to the microfuge tube and the present flexible pouch) were very comparable visually. Each had two relatively distinct bands at the same molecular weight, 27 kDa and 46 kDa (dimer), representing the known molecular weight of the GST protein (or dimeric form). There were no other eye-visible bands as compared to the waste fraction, which shows additional protein bands at higher molecular weights, for example. This demonstrates that the present flexible pouch devices may be used for protein purification and isolation.

The present flexible pouch devices may be configured for protein (or other cell culture product) isolation in relatively large volumes, such as, for instance, up to a liter or more of cell culture fluid. Multiple devices may be connected in parallel such that the fluidic circuitry is combined “downstream” of a cell lysis module. Alternatively, or additionally, a pouch may have several reservoirs for cell lysis. This may be advantageously used where one seeks to filter larger particulates.

Example 4

### Kit on a Pouch

This is a prophetic example. A flexible pouch device of the present invention is manufactured from a roll of flexible polymeric material, according to a predefined fluidic circuitry. The device is manufactured using fluid dispensing automation instrumentation to prefill selected reservoirs with desired fluids. The reservoirs are sealed, with a portion of the pre-filled reservoir having a seal that will burst with predetermined tensile force, such that the fluid is in fluid communication with a different reservoir. There are several reservoirs prefilled for a particular purpose, and fluid circuitry allows the fluids to flow to a predetermined area upon application of tensile force. For use, automation instrumentation applies force with rollers in a predetermined temporal pattern coordinating with the fluidic circuitry of the device.

### Example 5

### Biomarker Detection

This is a prophetic example. A flexible pouch device of the present invention is configured with fluidic circuitry for sorting a biomarker from a biological fluid obtained from an individual. The biomarker presence indicates a particular disease state. The biomarker is selected from among a cell, a protein, a nucleic acid, or a degradation product of any of the above. The disease state is selected from among a cancer, a neurological disease, and an infection. The cancer biomarker is selected from among a circulating tumor cell, a protein, and a nucleic acid. The neurological disease biomarker is selected from among a cell, a protein including but not limited to an abeta 1-42 protein or fragment or oligomer thereof, or other biomarker for a neurological disease selected from among Alzheimer's disease, Huntington's disease, Amylateral Sclerosis (“ALS” or Lou Gehrig's disease), a dementia, multiple sclerosis, and a disease caused by a prion. The biomarker for infection is selected from an infectious agent and a secondary pathogen or detectable marker of deleterious effect, and includes, but is not limited to, a virus, bacteria, a fungus, a prion and any other type of infectious agent. The virus may be an HIV virus, a hepatitis virus (of any type), a flu virus (of any type), a papilloma virus (HPV) of any type, a rabies virus, or any other viral infectious agent. The biomarker may be a portion of the organism or infectious agent so listed. For example, the biomarker may be a protein associated with a viral coat.

### Example 6

### Aptamer Screening

This is a prophetic example. A flexible pouch device of the present invention is configured with fluidic circuitry for use of an aptamer for detection of a rare molecule in a fluid sample. The aptamer is optionally associated with a detectable marker. The aptamer is exposed to a fluidic suspension under conditions for it to bind to its target. The aptamer and target are captured in a trapping station located within a reservoir in the present flexible pouch device. Non-target material is washed away with fluid (for example, buffer) applied using tensile force insufficient to dislodge the aptamer/target from the trapping station. This prophetic example may be used, for instance, to detect substances in urine, blood, or other bodily fluid. For example, one may detect trace amounts of cocaine or other illicit ingested pharmacological agents in urine. See, for example, Swensen, J. S. et al. Continuous, real-time monitoring of cocaine in undiluted blood serum via a microfluidic, electrochemical aptamer-based sensor. J. Am. Chem. Soc. doi:10.1021/ja806531z (2009), herein incorporated by reference.

### Example 7

### Testing for Analytes in Body Fluid

In this example, analytes such as a pharmaceutical or illicit drug are tested using flexible pouch and/or cartridges described herein. This is a prophetic example. For example, a flexible pouch device of the present invention is configured for fluidic circuitry so that an individual (such as a human or animal) may be monitored for drug presence or dosages. The present devices may be configured to detect or monitor medically prescribed dosages, pharmacokinetic, body or brain performance enhancing, illicit (methamphetamine, cocaine, marijuana (cannabinoids)) or endocrine related, such as glucose (insulin). For example, a flexible pouch device of the present invention is configured to provide prefilled reservoirs (or chambers) with reagents suitable for detecting pharmaceutical or pharmaceutical degradation or downstream metabolic agents, in a bodily fluid, such as blood or urine. A flexible pouch device is configured so that a blood (for example) sample is dispensed into a reservoir, and pre-filled pouches with suitable reagents are then permitted to open with manual tensile strength, such as the strength provided by a patients hands. The reagents when so combined with the bodily fluid provide a visible detection of whether the patient is properly dosed.

### Example 8

### Chemical Library Screening, Including Aptamer

This is a prophetic example. A flexible pouch device of the present invention is configured with fluidic circuitry for screening a library of chemicals for a particular purpose. For example, a library of aptamers may be screened against a protein target, such as by using a phage display. The aptamer/protein complexes may be analyzed to identify the aptamers so binding, and any binding characteristics, and the enriched aptamers may be subjected again to library screening. This may be performed in an iterative process to select aptameric moieties with particular characteristics, such as binding affinities or binding to particular epitopes on a protein moiety for example. A flexible pouch device of this example will have a reservoir for holding, and optionally culturing a phage display population of a predetermined protein, and inlet port or a prefilled chamber with the subject aptameric library to be so screened. Alternatively, one may have a reservoir holding an aptameric library to which is dispensed a desired protein (or other substrate for selection). The binding reaction may be aided with tensile motion applied in a reservoir to admix the aptamer library and protein (or other source).

### Example 9

### Genome Screening; DNA Analysis

This is a prophetic example. A flexible pouch of the present invention is configured with microfluidic circuitry and used in nucleic acid sorting. A sample of DNA is either placed within the pouch, or cells containing DNA are placed within the pouch, in fluid communication with reservoirs and channels for delivering reagents suitable to bind to particular DNA sequences (and optionally lyse cells to expose internal DNA if so desired or required). For example, DNA primers are used to bind to specific corresponding DNA sequences. The primers are applied to the reservoir containing the subject DNA (such as a genome or forensic sample). A wash fluid is added to the chamber to wash away unbound moieties. The primer/DNA is then exposed to several rounds of polymerase chain reaction, including applying reagent. The reagents are suitably mixed using automated instrumentation for applying tensile strength.

### Example 10

### Environmental Monitoring

This example is prophetic. A flexible pouch device of the present invention is configured with suitable materials and fluidic circuitry for environmental monitoring or analysis. While environmental fluid sample processing has much in common with aqueous fluid processing from biological fluids (above), modifications for field use include rugged material (e.g., made to withstand extremes in temperature, sunlight, salinity, or other environmental conditions), and use in the absence of reliable electricity. For example, a homeowner may wish to monitor drinking water, but collecting drinking water in a subject flexible pouch over a period of time, and analyzing once. Or, the present flexible pouch devices may be used for monitoring microbial species indicators for oil and gas drilling, where certain species are known to be associated with particular oil or gas containing geologic formations. Thus, one will select materials able to withstand sample application under these conditions. One may further configure the present flexible pouch devices so that manual (hand or hand-held tool) applied pressure is sufficient for fluidic flow in the desired way. Drinking or environmental water (such as saline or fresh water sources), soil (such as soil remediation), PCB or superfund site clean up monitoring, environmental radiation monitoring, repopulation (such as algae or krill) or other ecological purposes, as well as residential environmental monitoring (such as water, air or soil sample monitoring or analysis, including drinking or swimming pool water). One may use a prefilled device containing aptamers (for example, or other selective binding molecules) that selectively bind to heavy metals, such as mercury, lead, iron, or even gold or silver (for prospecting). One may monitor environmental toxins, such as arsenic, undue pharmaceutical environmental contamination, MBE's or other organic solvents. Acidification of oceanic areas, such as the continental shelf areas, may be performed with the inclusion of acidification indicators (for example, colorimetric strips) for example.

### Example 11

### Monitoring Biopharmaceutical Manufacturing

This is a prophetic example. The present flexible pouch devices may be used in the manufacture of biologicals for monitoring during the biological process. For example, one may collect protein from a separate bioreactor at various stages to monitor protein production for lot to lot variation. Vaccine manufacturing may also be monitored in this way. A variety of biologicals and biopharmaceutics can be monitored for quality assurance purposes using the present flexible pouch and/or cartridge devices.

### Example 12

### Point of Care, Diagnostic

This is a prophetic example. The present flexible pouch devices are configured suitably for various point of care blood panel analyses typically performed in a clinical laboratory. The present flexible pouch devices are configured so that a patient's blood is first deposited into a reservoir, and then, using tensile pressure, directed to flow to be partitioned in separate reservoirs. The blood sample so partitioned into individual reservoirs is then separately exposed to moieties used in such clinical laboratory practice, such as stains or dyes, or antibodies. Alternatively or additionally, the blood so partitioned may be exposed to alternative reagents better suited for the intended purpose, such as liver enzyme, blood sugar, thyroid, protein C or other blood moieties.

There are a wide variety of configurations and applications, and a skilled practitioner will ascertain these in view of the present disclosure. The present invention is not limited by the examples presented herein or the specific description.

1. A flexible fluid analysis device comprising:
(i) at least one reservoir in fluid communication with;
(ii) a first fluid channel, which is also in fluid communication with;
(iii) a mixing chamber, the mixing chamber also in fluid communication with;
(iv) a second fluid channel; and
(v) an outlet for draining fluids from the mixing chamber, the outlet in fluid communication with the second fluid channel;
wherein the flexible fluid analysis device comprises a unitary body.

2. The flexible fluid analysis device of claim 1, adapted for registration in an actuation instrumentation system for manipulating a fluid within the flexible fluid analysis device via application of one or more external forces and/or one or more internal forces.

3. The flexible fluid analysis device of claim 2, wherein the one or more external forces comprise at least one of a tensile force, a magnetic force, an acoustic force, an electrophoretic force and an optical force and the one or more internal forces comprise at least one of a pneumatic force and a hydraulic force.

4. The flexible fluid analysis device of claim 3, wherein the actuation instrumentation system is configured as a clamshell apparatus which holds the flexible fluid analysis device during operation.

5. The flexible fluid analysis device of claim 4, adapted for manipulation of magnetophoretic particles in the fluid by application of an external magnetic field.

6. The flexible fluid analysis device of claim 5, which is a flexible cartridge.

7. The flexible fluid analysis device of claim 5, which is a flexible pouch.

8. The flexible fluid analysis device of claim 6, wherein the flexible cartridge comprises four reservoirs, each in fluid communication to the mixing chamber via the first, and a second, a third and a fourth fluid channel, respectively.

9. The flexible fluid analysis device of claim 8, made from a material comprising at least one of polyethylene, polypropylene, polybutylene, polystyrene, polyvinylchloride, polytetrafluoroethylene, polycarbonate, polyethylene terephthalate, polyester, polyamide, polymethylmethacrylate, polyetheretherketone, nylon, fiber reinforced plastic, plastarch and polylactic acid.

10. The flexible fluid analysis device of claim 9, wherein the material is blow molded to form the flexible cartridge.

11. The flexible fluid analysis device of claim 10, wherein the mixing chamber also serves as a magnetic trapping region for magnetophoretic particles.

12. The flexible fluid analysis device of claim 11, wherein manipulating the fluid within the flexible fluid analysis device comprises carrying out a procedure for isolation or identification of a target species in the fluid.

13. The flexible fluid analysis device of claim 12, wherein the target species comprises at least one of a cell, a bacterium, a virus, a protein and a nucleic acid.

14. An automated actuation instrumentation system configured to manipulate a flexible fluid analysis device in order to carry out a procedure for isolation or identification of a target species in a fluid sample, wherein the flexible fluid analysis device is a flexible pouch or a flexible cartridge and comprises:
(i) at least one reservoir in fluid communication with;
(ii) a first fluid channel, which is also in fluid communication with;
(iii) a mixing chamber, the mixing chamber also in fluid communication with;
(iv) a second fluid channel; and
(v) an outlet for draining fluids from the mixing chamber, the outlet in fluid communication with the second fluid channel.

15. The automated actuation instrumentation system of claim 14, comprising a clamshell assembly for supporting the flexible pouch or the flexible cartridge while performing the procedure.

16. The automated actuation instrumentation system of claim 15, wherein manipulating the flexible fluid analysis device comprises applying one or more external forces to the flexible pouch or the flexible cartridge via at least one of a pump, a piston, a stepper motor, a pneumatic source and a roller.

17. The automated actuation instrumentation system of claim 16, further comprising a magnetic source for manipulating magnetic particles in the fluid sample as part of the procedure.

18. A flexible pouch device adapted for isolating a target species pre-labeled with a selectable binding agent from a fluid sample, the flexible pouch device including:
a pouch body including a fluidic circuit including at least one reservoir and at least one fluidic channel, the fluidic circuit adapted for
flow of the fluid sample through the fluidic circuit;
separation of the target species pre-labeled with the selectable binding agent from the fluid sample; and
collection of the separated target species within the fluidic circuit.

19. The flexible pouch device of claim 18, the target species comprises at least one of a cell, a bacterium, a virus, a protein and a nucleic acid.

20. The flexible pouch device of claim 19, wherein the target species is a cell, and the fluid sample is a cell suspension pre-treated with the selectable binding agent.

21. The flexible pouch device of claim 20, wherein the cell suspension is derived from a bodily fluid sample.

22. The flexible pouch device of claim 21, wherein the bodily fluid sample is a blood sample and the cell is a circulating tumor cell or a hematopoietic stem cell.

23. The flexible pouch device of claim 20 where the selectable binding agent is selected from an antibody and an aptamer.

24. The flexible pouch device of claim 20, wherein the selectable binding agent is an anti-CD34 selective binding agent and the cell is a stem cell.

25. The flexible pouch device of claim 20, adapted for isolating cells magnetophoretically, acoustophoretically, electrophoretically, or any combination thereof.

26. The flexible pouch device of claim 20, adapted for microfluidic flow.

27. The flexible pouch device of claim 25, further comprising a magnetically responsive trapping station.

28. An apparatus for creating a fluidic circuit from a featureless bag, the apparatus comprising:
(i) one or more molds and/or clamps which create the fluidic circuit upon engagement with the featureless bag; and
(ii) one or more actuators for manipulating the fluidic circuit, the manipulation comprising valving and pumping a fluid sample within the fluidic circuit.

29. The apparatus of claim 28, further comprising one or more external forces for manipulating a target species within the fluidic circuit, the one or more external forces comprising at least one of a tensile force, a magnetic force, an acoustic force, an electrophoretic force and an optical force and one or more internal forces comprising at least one of a pneumatic force and a hydraulic force.

30. A process for manufacturing a plurality of flexible pouch devices from one or more sheets of polymeric material, the method comprising:
(i) arranging the one or more sheets of polymeric material so that there is an overlapping region of the polymeric material; and
(ii) applying opposing plates with premilled molds to the overlapping region in order to form at least one flexible pouch device of the plurality of flexible pouch devices;
wherein each flexible pouch device comprises at least one fluidic circuit including at least one reservoir in fluid communication with another reservoir.
